(19) United States
(12) Patent Application Publication
(10) Pub. No.: US 2024/0400488 A1
(43) Pub. Date: Dec. 5, 2024

(54) EXTRACTION AND PURIFICATION OF NATURAL FERULIC ACID FROM BIOMASS AND CONVERSION TO VANILLIN

(71) Applicant: Spero Renewables, LLC, Goleta, CA (US)

(72) Inventors: Ian M. KLEIN, Goleta, CA (US); Shou ZHAO, Goleta, CA (US); Sean PRAGER, Goleta, CA (US); Susanna SEPPÄLÄ, Goleta, CA (US)

(21) Appl. No.: 18/696,868

(22) PCT Filed: Sep. 30, 2022
(86) PCT No.: PCT/US2022/077314

§ 371 Date: Mar. 28, 2024

Related U.S. Application Data
(60) Provisional application No. 63/251,365, filed on Oct. 1, 2021.

(51) Int. Cl. C07C 51/47

(52) U.S. Cl. CPC C07C 51/47

(57) ABSTRACT

A process for a reactive extraction and subsequent purification of organic molecules from biomass includes extracting one or more products from the biomass using an extraction solvent to solvate the products, contacting the biomass with a reactant during the extracting and separating the liquid products from solid products. Liquid products include ferulate or ferulic acid and are purified to varying levels of purity before conversion of ferulic acid to vanillin by biological means.

### CROSS-REFERENCE TO RELATED APPLICATIONS

This application claims priority to U.S. Provisional Patent Application No. 63/251,365 filed on Oct. 1, 2021, and entitled “EXTRACTION AND PURIFICATION OF NATURAL FERULIC ACID FROM BIOMASS AND CONVERSION TO VANILLIN,” which is incorporated herein in its entirety by reference.

### STATEMENT REGARDING GOVERNMENTALLY SPONSORED RESEARCH OR DEVELOPMENT

Not applicable.

### BACKGROUND

Consumer demands for natural products and ingredients have driven the industry to seek new sources of natural vanillin, while vanillin derived from synthetic, non-natural sources such as petrochemicals and eugenol have seen a decrease in demand. Vanilla extracts obtained from the vanilla bean cannot keep pace with rising demand and human population; it is limited to certain geographic locations and results in harmful deforestation. The most viable solution to obtaining natural vanillin is through fermentation of natural ferulic acid which can be sourced from a suitable natural/biomass feedstock. Carbon 13 NMR of vanillin (the active molecule in vanilla flavoring) is used to determine the source of vanillin by the ratio of ¹³C and ¹²C at the eight carbons of the vanillin molecule, with the carbons of the aldehyde and the methoxy group determined to be the most important. Synthetic sources such as guaiacol can be discriminated from natural vanillin sourced from vanilla beans and ferulic acid, with thresholds dependent on the experimental setup.

Much of the supply of natural ferulic acid is extracted during the processing of rice bran oil. Ferulic acid is also contained in agricultural biomass such as miscanthus, corn byproducts (fiber, bran, stover, fines, gluten feed, etc.), rice, wheat, beets, beet fiber, beet pulp, and other crops. Typical methods for extracting ferulic acid include alkali extraction and enzymatic processes. Alternatively, ferulic acid is extracted as several ferulic acid phytosterol esters such as γ-oryzanol. Excess alcohol extraction has been used to remove lignin from biomass, and similar extractions have been used to extract ferulic acid esters. For example, in the presence of methanol, the extract contains methyl ferulate (methyl 3-(4-hydroxy-3-methoxyphenyl) acrylate), while in the presence of ethanol the extract contains ethyl ferulate (ethyl 3-(4-hydroxy-3-methoxyphenyl) acrylate). Additional processes for recovering ferulic acid from biomass are described in WO2018/195422 to Abu-Omar, et al. and WO2021/011810 to Stair, et al., which are both incorporated herein in its entirety by reference.

### SUMMARY

In some aspects, a process for a reactive extraction and subsequent purification of organic molecules from biomass comprises extracting one or more products from the biomass using an extraction solvent to solvate the products, contacting the biomass with a reactant during the extracting, diluting reaction products with a solvent, diluting reaction products with an organic solvent to precipitate one or more impurities, separating the liquid products from solid products, recovering the one or more products, performing a hydrolysis reaction on the liquid product to convert ferulate esters to ferulic acid, performing a hydrolysis on the liquid product to hydrolyze one or more impurities, selectively dissolving one or more products into one or more organic solvents to remove one or more impurities, performing liquid-liquid extractions to selectively transfer one or more products to an organic solvent, performing ultrafiltration or nanofiltration to remove impurities from the one or more products to produce a filtered extract, extracting oils in the filtered extract using adsorption to produce a de-oiled extract, extracting oils in the filtered extract using contact with an organic solvent to remove the oils from other products, adjusting the pH of the extract solution to selectively precipitate one or more products which can then be removed by filtration, performing transesterification or hydrolysis on the de-oiled extract, and performing liquid chromatography to further purify the ferulic acid, coumaric acid, ferulate, coumarate, or a combination thereof. Performing adsorptive purification to further purify the ferulic acid, coumaric acid, ferulate ester, coumarate ester, or a combination thereof. Dissolving and recrystallizing the product to further purify the ferulic acid, coumaric acid, ferulate ester, coumarate ester, or a combination thereof. The one or more products comprise extracted organic molecules comprising a ferulate ester or ferulic acid or a coumarate ester or coumaric acid, and the one or more products are separated from the biomass as a liquid extract. The ferulate ester or the coumarate ester can be reacted in a transesterification or hydrolysis to produce ferulic acid, coumaric acid, ferulate ester, coumarate ester, or a combination thereof. Ferulic acid, coumaric acid, ferulate ester, coumarate ester, or a combination thereof are purified to produce one or more purified products. Products including ferulate or ferulic acid and are purified to varying levels of purity before conversion of the ferulic acid to vanillin by biological means.

### BRIEF DESCRIPTION OF THE DRAWINGS

For a more complete understanding of the present disclosure, reference is now made to the following brief description, taken in connection with the accompanying drawings and detailed description:

FIG. 1 shows a schematic flow chart of a process for the purification of ferulic acid or ferulate from an extraction mixture or ferulic acid or ferulate dissolved in an aqueous or organic solvent according to some embodiments.

FIG. 2 shows a schematic flow chart of a process for the purification of ferulic acid or ferulate from an extraction mixture (9A) or ferulic acid or ferulate dissolved in an aqueous or organic solvent and conversion to vanillin according to some embodiments.

FIG. 3 shows a schematic flow chart of a process for the purification of ferulic acid (FA) through concentration, selective dissolution, silica gel column and hot water filtration according to some embodiments.

FIG. 4 shows a schematic flow chart of a process for the purification of ferulic acid through concentration, selective dissolution, hexane extraction and hot water filtration according to some embodiments.

FIG. 5 shows a schematic flow chart of a process for the purification of ferulic acid through concentration, selective dissolution, hexane extraction/precipitation and hot water filtration according to some embodiments.

FIG. 6 shows a schematic flow chart of a process for the purification of ferulic acid through concentration, selective dissolution, hexane extraction/precipitation, silica gel column and hot water filtration according to some embodiments.

FIG. 7 shows a schematic flow chart of a process for the purification of ferulic acid through concentration, silica gel column and hot water filtration according to some embodiments.

FIG. 8 shows a schematic flow chart of a process for the purification of ferulic acid through concentration, cool grinding, hot water filtration, ethyl acetate extraction, hexane extraction and hot water filtration according to some embodiments.

FIG. 9 shows a schematic flow chart of a process for the purification of ferulic acid through concentration, selective dissolution, hot water filtration, hexane extraction and hot water filtration according to some embodiments.

FIG. 10 shows a schematic flow chart of a process for the process flow diagram following extraction example #1.c, followed by purification example #5.

### DETAILED DESCRIPTION

It is understood at the outset that although an illustrative implementation of one or more embodiments are provided below, the disclosed systems and methods may be implemented using any number of techniques, whether currently known or in existence. The disclosure should in no way be limited to the illustrative implementations, drawings, and techniques illustrated herein below, including the exemplary designs and implementations illustrated and described herein, but may be modified within the scope of the appended claims along with their full scope of equivalents. Thus, while multiple embodiments are disclosed, still other embodiments will become apparent to those skilled in the art from the following detailed description. As will be apparent, certain embodiments, as disclosed herein, are capable of modifications in various aspects without departing from the spirit and scope of the claims as presented herein. Accordingly, the detailed description herein below is to be regarded as illustrative in nature and not restrictive.

Ferulic acid (3-(4-hydroxy-3-methoxy-phenyl) prop-2-enoic acid, 3-(4-hydroxy-3-methoxyphenyl) acrylic acid) is a powerful antioxidant used in consumer products and pharmaceuticals. Ferulic acid is also used to produce natural vanillin, the active molecule/ingredient in vanilla flavoring. The flavor and fragrance industry transforms ferulic acid to natural vanillin using enzymatic processes. Natural vanillin produced from natural ferulic acid is of particular importance due to the volatility, high cost, and scarcity of natural vanilla bean extracts.

The extracted ferulate and coumarate can be regarded as natural, for example following European 1334/2008 and US Food and Drug administration (FDA) 21CFR101.22 regulations regarding natural labeling. Extracted ferulate esters can be hydrolyzed to ferulic acid and isolated as a pure, crystalline solid or as an aqueous solution. Extracted ferulic acid can be isolated as a pure, crystalline solid or as an aqueous solution. Additional cinnamic acids, sugars, oils, and fatty acids are also extracted during the process and can be isolated as coproducts. This application describes a process for producing ferulic acid from biomass, where said process may comprise any number of the steps summarized herein, where such steps can comprise pretreatment of agricultural biomass, reactive extraction of ferulic acid and coumaric acid and ferulate ester (ferulic acid esters of the variety methyl-, ethyl-, propyl-, butyl-, or any variation thereof) and coumarate ester, purification of the resulting ferulate ester, coumarate ester, ferulic acid, and coumaric acid using filtration methods and chromatography, optional conversion of ferulate and coumarate esters to ferulic acid and coumaric acid, purification of ferulic acid by ion exchange, adsorptive purification, selective dissolution into organic solvents, liquid-liquid separations with aqueous and organic solvents, washing of solid or semi-solid products with organic solvents, pH adjustments to precipitate impurities or change the solubilities of extracted compounds, chromatography, crystallization and recovery of solid ferulic acid and coumaric acid.

The ferulic acid and ferulate purification methods described herein can be applied to any ferulic acid or ferulate containing process stream in addition to purification of ferulic acid and ferulate extracted from biomass as described herein. For example, the ferulic acid and ferulate purification methods described herein can be used to purify ferulic acid or ferulate which has been extracted from biomass using alternative extraction methods to those described herein. The ferulic acid and ferulate purification methods described herein could also be used to purify ferulic acid or ferulate contained in a variety of biomass process streams, including but not limited to purification of ferulic acid or ferulate from waste streams of corn processing such as the ferulic acid containing aqueous nejayote process stream from corn tortilla processing, olive mill process streams and wastewater, wheat processing process streams and wastewater, or wheat distillers grains processing streams and wastewater.

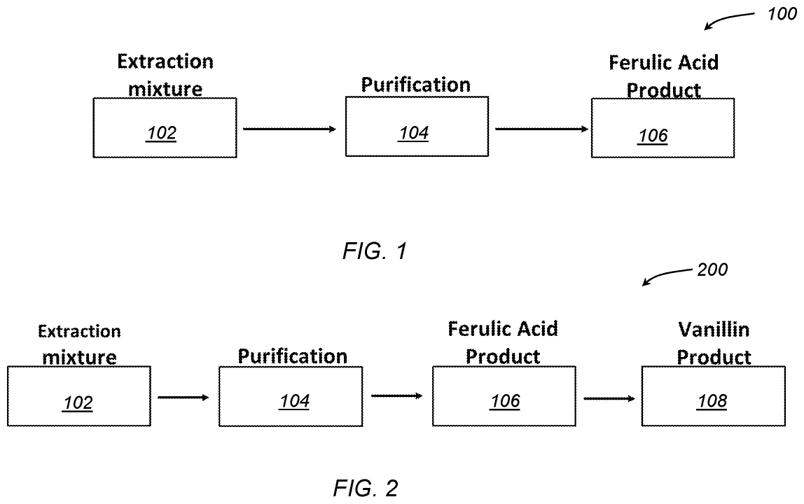

FIG. 1 shows a schematic flow chart of a process 100 for the purification of ferulic acid or ferulate. The process 100 can comprise an extraction step 102 from an extraction mixture, or an extraction of ferulic acid or ferulate dissolved in an aqueous or organic solvent. A purification step 104 can be used on the mixture and can include any number of processes in any order for the purification of a ferulic acid or ferulate containing solution including concentration, selective dissolution, silica gel adsorption, hot water filtration, solvent based oil extraction, solvent based precipitation, selective adsorption, ultrafiltration, and nanofiltration, each of which is described in more detail herein. The final step 106 can include the production of a purified ferulic acid product according to some embodiments. Depending on the purification methods used in the purification step 104, the purity of the ferulic acid product in step 106 can range from about 15 wt. % pure ferulic acid to 100 wt. % pure ferulic acid.

FIG. 2 shows another schematic flow chart of a purification process 200. The process 200 can be similar to the process 100, and similar steps may be the same or similar. The process 200 can start with an extraction mixture at step 102. A purification step 104 for the purification of ferulic acid or ferulate from the extraction mixture or ferulic acid or ferulate dissolved in an aqueous or organic solvent can then be carried out. The purification step 104 can include any number of processes in any order for the purification of a ferulic acid or ferulate containing solution including concentration, selective dissolution, silica gel adsorption, hot water filtration, solvent based oil extraction, solvent based precipitation, selective adsorption, ultrafiltration, and nanofiltration to produce a purified ferulic acid product at step 106 according to some embodiments. Depending on the purification methods used the purification step 106, the purity of the ferulic acid product can range from 15 wt. % pure ferulic acid to 100 wt. % pure ferulic acid. The ferulic acid from the purification step 106 can then be converted to vanillin in step 108 according to some embodiments.

In some aspects, a processing step to produce the extraction mixture or aqueous solution can include ferulate ester extraction, which can include ferulate ester reactive extraction. In some embodiments, a process for reactive extraction of ferulate and coumarate esters can include a reaction step and/or extraction step for the extraction of ferulate and coumarate esters from biomass. The reaction step and extraction step can occur simultaneously as a reactive extraction step. Any suitable biomass can be used as a starting feedstock for the reaction and extraction steps. Various exemplary biomass feedstocks include, but are not limited to, miscanthus, corn bran, corn fiber, corn gluten feed, dried distillers grains (DDG), dried distillers grains with solutes (DDGS), distillers grains, corn stover, corn gluten meal, beet fiber, rice hulls, rice bran, wheat bran, or other wheat derived feedstocks, and/or other agricultural residues containing ferulate linkages. Although corn fiber or other types of biomass used in the reactive extraction process can be dried prior to the reactive extraction step, a drying step is not required. Advantageously, it is noted that the presence of water in wet corn fiber or other types of wet biomass does not have a negative effect on the reactive extraction presented herein. It is noted that use of wet corn fiber or other wet biomass can reduce processing costs and energy use associated with drying biomass.

In some embodiments a pretreatment step can be used prior to the reaction and extraction or reactive extraction steps to remove certain components of the biomass feedstock.

The reaction step, which can occur prior to or simultaneously with the extraction step, can occur with a base. The extraction step can occur in the presence of a solvent. The products can comprise at least one of: ferulic acid, ferulate (ferulic acid ester), coumaric acid, and coumarate (coumaric acid ester). Exemplary ferulate esters can include but are not limited to: methyl ferulate, ethyl ferulate, propyl ferulate, and butyl ferulate. Exemplary coumarate esters can include but are not limited to methyl coumarate, ethyl coumarate, propyl coumarate, and butyl coumarate. Coproducts are also extracted and include oils, which are typically comprised of oleic acid, oleate (oleic acid ester), linoleic acid, and linoleate (linoleic acid ester). Additional coproducts can include but are not limited to proteins, amino acids, carbohydrates, carbohydrate derived materials, sugars, salts, and fiber.

The extraction and reaction steps of the process can occur with the aid of an extraction solvent. In some embodiments, the extraction solvent can comprise an alcohol (e.g., an organic alcohol), water, or a mixture of an alcohol and water. When water is present, at least a portion of the water can be provided by, or as part of, the biomass. As noted in the examples disclosed herein, ferulic acid and coumaric acid are observed as reaction products when water is used as the reactive extraction solvent. When mixtures of alcohol and water are used as the reactive extraction solvent the reaction products can include ferulic acid and coumaric acid, and in some cases the reaction products can include mixtures of ferulic acid, ferulate ester, coumaric acid, and coumarate ester. While not wishing to be limited by theory, it has been noted that the identity of the ferulate ester is directly linked to the chosen alcohol in the solvent. For example, the use of methanol yields methyl ferulate and the use of ethanol yields ethyl ferulate. Esters of coumaric acid are simultaneously extracted.

In some embodiments, a reaction step can also be carried out using a base. This reaction step can be carried out at the same time as the extraction. For example, base can be added to the solvent to enhance solubility and rates of reaction and extraction at lower reaction temperatures. Bases include any first or second group hydroxides such as sodium hydroxide, potassium hydroxide, calcium hydroxide, carbonates, bicarbonates, and ammonium in concentrations of about 0 to about 6 N (molar equivalents of base per liter of solvent).

Various process designs can be used to carry out the extraction and/or reaction steps. Any suitable reactor configuration capable of contacting the biomass feedstock with the solvent and/or base can be used. For example, the reactive extraction of ferulic acid and ferulate esters can occur in a stirred or unstirred batch reactor, continuous stirred-tank reactor (CSTR), packed bed reactor (PBR), or other reactor types known to those skilled in the art.

To improve or optimize the yield of ferulic acid or ferulate ester, several variables can be manipulated, including the concentration of extraction aid or base, extraction solvent to biomass ratio, the reactive extraction temperature, the reactive extraction time, the specific biomass, the extraction solvent composition, and/or the reactor equipment. Several sets of operating conditions are hereinafter enumerated, but other regimes of operating conditions are possible and included as part of this disclosure.

In some embodiments the reactive extraction is carried out between 5° C.-200° C. with a solvent to biomass volumetric ratio of 1 to 30, with a sodium hydroxide or base concentration of 0.01 M-6 M, and a reaction time of 0.1-24 hours.

In some embodiments, residual biomass such as corn fiber can be removed from the reactive extraction product by filtration, centrifugation, twin roll press, screw press, filter press, and vacuum filtration. Advantageously, removal of residual solid biomass can produce a solid material suitable for animal feed while simultaneously increasing the purity of the ferulic acid or ferulate ester in the reactive extraction product.

In some embodiments, the reactive extraction can extract coproducts to ferulic acid and ferulate ester including but not limited to proteins, arabinoxylans, heteroxylans, hemicellulose, cellulose, carbohydrates, xylose, glucans, fatty acids, fatty acid esters, and derivatives of such. In embodiments where the reactive extraction solvent contains some amount of water, an organic solvent can be added to the reaction mixture during the reactive extraction or after the reactive extraction has completed to precipitate coproducts from the reaction mixture as solids, semi-solids, or oils. In some embodiments the organic solvent used to precipitate coproducts can be hexanes, acetates (e.g., ethyl acetate), ethanol, aliphatic alcohols, or other organic solvents. The coproducts precipitated by addition of the organic solvent can then be removed from the reaction mixture along with residual solids of corn fiber or biomass by filtration, centrifugation, twin roll press, screw press, filter press, and vacuum filtration. Advantageously, precipitation of coproducts and removal of said coproducts with residual corn fiber or biomass solids can increase the nutritional value of the residual corn fiber or biomass while simultaneously increasing the purity of the ferulic acid or ferulate ester in the reaction mixture. Advantageously, precipitation of coproducts and removal of said coproducts with residual corn fiber solids or biomass solids can decrease the viscosity of the reaction product. In some embodiments, the ferulic acid or ferulate ester rich liquid phase can then be concentrated to yield a water rich mixture which can undergo a liquid-liquid extraction to selectively extract ferulic acid or ferulate ester from the aqueous rich solution to the organic solvent, producing a ferulic acid or ferulate ester dissolved in the organic solvent where the purity of ferulic acid or ferulate ester is higher in the organic solvent than in the water rich liquid it was extracted from. Impurities such as sugars, proteins, carbohydrates, and salts remain in the water rich liquid phase. The organic solvent used in the liquid-liquid extraction can include but is not limited to hexanes, toluene, diethyl ether, ethyl acetate, or any organic solvent which is immiscible with water. As an alternative to the liquid-liquid extraction, the ferulic acid or ferulate ester rich liquid phase can be concentrated to yield a solid, semi-solid, or oily product from which the ferulic acid or ferulate ester can be selectively extracted into an organic solvent, increasing the purity of the ferulic acid or ferulate ester. The organic solvent used in the selective dissolution can include but is not limited to hexanes, toluene, diethyl ether, ethyl acetate, or other common organic solvents.

In some embodiments, coproducts to ferulic acid and ferulate ester including but not limited to proteins, arabinoxylans, heteroxylans, hemicellulose, cellulose, xylose, glucans, and derivatives remain in the liquid reactive extraction product along with ferulic acid or ferulate ester following removal of solid residual corn fiber or biomass from the liquid product of the reactive extraction. In some cases, the coproducts can be decomposed or partially decomposed into coproducts including but not limited to glucose, xylose, amino acids, derivatives of such, or otherwise made water soluble by performing a hydrolysis reaction on the liquid reactive extraction product. This hydrolysis can be catalyzed by addition of acid or base to the liquid reactive extraction product and can optionally be heated. Suitable bases for the hydrolysis include but are not limited to any first or second group hydroxides such as sodium hydroxide, potassium hydroxide, calcium hydroxide, carbonates, bicarbonates, and ammonium in concentrations of about 0 to about 6 N (molar equivalents of base per liter of solvent), or sufficient base content to increase the pH to a pH of up to 13. Suitable acids for the hydrolysis include but are not limited to hydrochloric acid, nitric acid, phosphoric acid, sulfuric acid, acetic acid, and combinations thereof in concentrations of about 0 to 6 N (molar equivalents of acid per liter of solvent), or sufficient acid to decrease the pH to a pH of as low as 1. The hydrolysis mixture can be heated to a temperature of 20° C.-120° C. for a time period of 0-12 hours to complete the hydrolysis. Advantageously the reaction product following hydrolysis can have a decreased viscosity. In some embodiments the liquid reaction product following hydrolysis can be extracted with an organic solvent in a liquid-liquid extraction to yield a ferulic acid or ferulate ester rich organic phase, increasing the purity of the ferulic acid or ferulate ester. Impurities such as sugars, proteins, carbohydrates, and salts remain in the aqueous phase. The organic solvent used in the liquid-liquid extraction can include but is not limited to hexanes, toluene, diethyl ether, ethyl acetate, or any organic solvent which is immiscible with water. In other embodiments the liquid reaction product following hydrolysis can be concentrated to yield a solid, semi-solid, or oily product from which the ferulic acid or ferulate ester can be selectively dissolved into an organic solvent, increasing the purity of the ferulic acid or ferulate ester. The organic solvent used in the selective extraction can include but is not limited to hexanes, toluene, diethyl ether, ethyl acetate, or other common organic solvents.

In some embodiments, ferulic acid produced in the reactive extraction can be purified by sublimation following the reactive extraction. In other embodiments, ferulic acid can be purified by sublimation before or after any of the processing steps following the reactive extraction. In some embodiments, ferulic acid is sublimed by heating a crude ferulic acid solution with or without applying vacuum and condensing gaseous ferulic acid to produce a purified solid ferulic acid product.

The processes and methods disclosed herein provide a number of new improvements around the recovery of products including ferulic acid and related compounds. For example, the treatment of the biomass extract in water after removal of the fiber with base to hydrolyze arabinoxylan is new. It allows for phase separation via liquid-liquid extraction with organic solvent such as ethyl acetate. This selective dissolution is a new way to recover ferulic acid from a mixture comprising carbohydrates and water-soluble by-products. Otherwise, the high viscosity of the arabinoxylan (corn gum) in the extract can hinder processing and further purification.

Similarly, washing the water extract with an alcohol such as ethanol to precipitate arabinoxylans and other carbohydrates is new. This allow for the use of fiber that is nutrient rich and suitable for feed to be used in the process and followed by liquid-liquid extraction to give a ferulic acid organic layer for further purification. Without this step, the extract can form an emulsion and may not be able to be processed further. Additional new elements include hot water filtration to recover ferulic acid from other phenols. Further the separation steps as described herein are also new. Each of these elements is new in the context of using the obtained ferulic acid to produce vanillin.

In some embodiments, a biomass extract containing ferulic acid or ferulate or an aqueous or organic process stream containing ferulic acid or ferulate can undergo a range of purification steps as outlined in FIGS. 1 and 2, to produce a ferulic acid product where the purity of the ferulic acid product can range from 15 wt. % pure ferulic acid to 100 wt. % pure ferulic acid. When the ferulic acid product has a purity of less than 100 wt. % purity, the contaminants can be grouped into four categories of Group 1, Group 2, Group 3, and Group 4 contaminants as follows. Group 1 contaminants can comprise lignin or lignin derived materials and can include any combination of, some of, or one of the following: lignin, polyphenols, coumaric acid, substituted phenols, or lignin derived materials. While not wishing to be limited by theory, it is noted that Group 1 contaminants can often be characterized by UV-Visible Spectroscopy and often have a Lamda max of about 200 nanometers (nm) and a local maximum absorbance at about 240 nm or about 320 nm, or Group 1 contaminants can be assayed as Total Phenolic Content using several methods. Group 2 contaminants can comprise carbohydrates or carbohydrate derived materials and can include any combination of, some of, or one of the following: carbohydrates, hemicellulose, cellulose, arabinoxylans, heteroxylans, xylans, glucans, sugars, and carbohydrate derived materials. Group 3 contaminants can be water soluble and can include any combination, some of, or one of the following: proteins, amino acids, nitrogen containing compounds, and salts. Group 4 contaminants are often characterized as oils and can include any combination of, some of, or one of the following: fatty acids, oleic acid, linoleic acid, fatty acid esters, sterols, and phytosterols.

Ferulic acid which is extracted from biomass as described herein can be converted to vanillin (e.g., as in step 108 of FIG. 2) using a number of biological conversion techniques to produce a vanillin product that can be regarded as natural, for example following European 1334/2008 and US Food and Drug administration (FDA) 21CFR101.22 regulations regarding natural labeling. Advantageously the methods of ferulic acid reactive extraction described herein produce a ferulic acid product which can be directly converted to vanillin without need for further purification of the ferulic acid following reactive extraction from biomass. Additionally, the ferulic acid product of any of the purification steps described herein can be converted to vanillin. This may be advantageous when it is desirable to convert a ferulic acid of higher purity to vanillin than the ferulic acid produced in the initial reactive extraction step. Ferulic acid conversion to vanillin directly after the reactive extraction of ferulic acid from biomass or ferulic acid conversion to vanillin following any of the purification steps described herein can be achieved by reaction with many biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and other microorganisms and biological agents can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system.

In some embodiments a ferulic acid product where the purity of the ferulic acid product can range from 15 wt. % pure ferulic acid to 100 wt. % pure ferulic acid can be further converted to a vanillin product as illustrated in step 108 of FIG. 2. The conversion of ferulic acid to vanillin can occur by reaction with many biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and other microorganisms and biological agents can be used to complete the conversion of ferulic acid to vanillin. When the ferulic acid product has a purity of less than 100 wt. % purity, the contaminants can be grouped into four categories of Group 1, Group 2, Group 3, and Group 4 contaminants as previously described. When ferulic acid of less than 100% purity containing any combination of Group 1-4 contaminants is converted to vanillin using biological agents, the vanillin product can be produced as a unique mixture where the vanillin product is produced as a mixture of any combination of Group 1-4 contaminants along with any unreacted ferulic acid or bioproducts including but not limited to guaiacol, vanillic acid, vanillyl alcohol, 4-vinylguaiacol, protocatechuic acid, catechol, or any combination thereof.

As described herein, a number of purification steps (e.g., purification steps 104 in FIGS. 1 and/or 2) that can remove specific impurities from the ferulic acid containing products following the reactive extraction step can be used in some embodiments. The purification steps which can be applied to the reactive extraction liquid product include but are not limited to: concentration, selective dissolution, adsorption, hot water filtration, organic solvent extraction/precipitation, sublimation, cation exchange, anion exchange, and resin adsorption. These purification steps and other purification steps known to those skilled in the art can be used in different orders or combinations which are obvious to those skilled in the art to produce ferulic acid with high purity (>95%).

In some embodiments, concentration can be applied to the reactive extraction liquid product, alone or in combination with other purification processes. Concentration is a process method in which the solvents or volatile fractions of the liquid product mixture are removed through evaporation. Concentration methods include but are not limited to distillation and rotary evaporation. In some embodiments, a solid material including but not limited to activated carbon, celite, silica, alumina, diatomaceous earth, or mixtures of one or more of these materials can be added during concentration of the crude extract mixture. In some embodiments, spray drying can be used to further concentrate extract solutions, forming a powder-like solid before addition to the organic solvent or ethyl acetate solution used for selective dissolution.

In some embodiments, selective dissolution can be applied to the reactive extraction liquid process alone or in combination with other purification processes. Dissolution is a process method in which the ferulic acid or ferulate ester containing mixture before or after optional concentration is dissolved in a minimum amount of solvents. Suitable solvents for this step include but are not limited to ethanol, water, acetone, hexane, ethyl acetate, diethyl ether or the mixture of at least two above mentioned solvents. In some embodiments the pH of the liquid product mixture is adjusted to between pH 2-5 with acid including, but not limited to sulfuric acid, hydrochloric acid, nitric acid, lactic acid, acetic acid, or other acids. The acidified mixture can be poured into a solvent including but not limited to ethanol, acetone, hexane, ethyl acetate, diethyl ether, or the mixture of at least two above mentioned solvents. The mixture can be stirred and agitated at 0-100° C. for 10-1000 min., selectively dissolving the ferulic acid or ferulate ester component in the organic solvent fraction, while water-soluble impurities precipitate or remain soluble in an aqueous phase. The ferulic acid or ferulate ester rich organic layer can be collected and concentrated for subsequent purification. Relevant process alternatives to selective dissolution include but are not limited to cation exchange, anion exchange, simulated moving bed chromatography, liquid chromatography, counter current chromatography, ultrafiltration, nanofiltration, rotating disk ultra or nano filtration, or sublimation.

In some embodiments silica gel adsorption can be applied to the reactive extraction liquid process alone or in combination with other purification processes. Silica gel adsorption is a purification method in which ferulic acid or ferulate ester can be separated from impurities through differences in polarity or molecular weight. This step can be used to remove the majority of corn oils, fatty acids, fatty acid esters, sterols, inorganic compounds, lignin-derived phenolic compounds, sugars, xylans, amino acids and other high-polarity impurities. After the silica gel column is packed, a ferulic acid or ferulate containing mixture which has been dissolved in a minimal amount of solvent can be applied on the top of the column and the column can be flushed using the mobile phase where the mobile phase can be a mixture of acetone and hexane, ethyl acetate and hexane, ethanol and hexane, or similar solvent combinations known to those skilled in the art. The volume ratio of the two components in the mobile phase ranges from 100:0.1 to 0.1:100. Product fractions can be collected from the column as the mobile phase passes through the column. The ferulic acid or ferulate ester containing fractions can be combined and concentrated for further purification. Alternatively, alumina, diatomaceous earth, activated carbon or similar materials can be used as a direct replacement for silica using similar methods. Relevant process alternatives to silica gel adsorption include but are not limited to cation exchange, anion exchange, simulated moving bed chromatography, liquid chromatography, counter current chromatography, ultrafiltration, nanofiltration, rotating disk ultra or nano filtration, or sublimation.

In some embodiments hot water filtration can be applied to the reactive extraction liquid process alone or in combination with other purification processes. Hot water filtration is a purification method in which ferulic acid or ferulate ester can be separated from impurities based on solubility differences of the ferulic acid or ferulate ester and the impurities in water. Representative impurities can include but are not limited to corn oils, fatty acids, fatty acid esters, sterols, inorganic compounds, lignin-derived phenolic compounds, sugars, xylans, amino acids. The ferulic acid or ferulate ester containing mixture can be added to a certain amount of water. In some embodiments the pH of the water is in the range of 2-7, while the ferulic acid or ferulate containing mixture and water weight ratio ranges from 1:1 to 1:100. The mixture of the ferulic acid or ferulate ester containing solution and water can be heated to boiling to achieve a clear or yellow hued liquid portion and a fraction of precipitate. The liquid portion can be filtered when hot, followed by cooling to room temperature. The filtered solution can be left at 0-30° C. for 1-100 hours to allow ferulic acid or ferulate ester to precipitate from the solution. When the precipitation of ferulic acid or ferulate ester is finished, the mixture can be filtered to collect the ferulic acid-containing solid. Relevant process alternatives to hot water filtration include but are not limited to cation exchange, anion exchange, simulated moving bed chromatography, liquid chromatography, counter current chromatography, ultrafiltration, nanofiltration, rotating disk ultra or nano filtration, or sublimation.

In some embodiments, a solvent based oil extraction can be applied to the reactive extraction liquid process alone or in combination with other purification processes. Solvent based oil extraction is a purification method in which corn oil, fatty acids, fatty acid esters, sterols, phytosterols, lipids, or other non-polar impurities can be removed from the ferulic acid or ferulate ester containing mixture. The ferulic acid or ferulate ester and corn oil, fatty acid, fatty acid esters, sterol, phytosterol, lipid, or other non-polar impurity containing mixture can be dissolved in a minimal amount of solvent. Suitable solvents can include but are not limited to ethanol, acetone, ethyl acetate, or the mixture of at least two above mentioned solvents. This solution can be added to a stirred secondary solvent of heptane, toluene, petroleum ether, hexane, or similar solvent in 1:1 to 1:100 volume ratio. The mixture can be stirred at 0-100° C. for 0.1-24 hours. The secondary solvent rich liquid portion can be collected as a separate phase from a semi-solid, solid, or oil like residue which contains the majority of ferulic acid or ferulate ester. The secondary solvent rich liquid portion can contain corn oil, fatty acids, fatty acid esters, sterols, phytosterols, lipids, or other non-polar impurities. The ferulic acid or ferulate ester rich sample can be used for further purification. In some embodiments the secondary solvent extraction can be repeated another 2-3 times to remove additional corn oil, fatty acids, fatty acid esters, sterols, phytosterols, lipids, or other non-polar impurities from the ferulic acid or ferulate ester. Relevant process alternatives to solvent based oil removal include but are not limited to cation exchange, anion exchange, simulated moving bed chromatography, liquid chromatography, counter current chromatography, ultrafiltration, nanofiltration, rotating disk ultra or nano filtration, sublimation, or resin adsorption to resins including but not limited to derivatives of polystyrene divinylbenzene resins.

Under certain conditions, the solvent based oil extraction process can also precipitate a certain amount of ferulic acid solids (10-50 wt. % yield). These solids have relatively high ferulic acid purity (up to 90 wt. %). Three methods can be used to precipitate additional high-purity ferulic acid solids from the solvent based extraction/precipitation mixture, including sonicating or stirring the mixture, increasing the temperature of the mixture and adding a certain amount of a solvent such as acetone, ethanol or ethyl acetate to the solution.

In some embodiments, a solvent based precipitation can be applied to the reactive extraction liquid process alone or in combination with other purification processes. Solvent based precipitation can be used as a purification step where addition of a solvent to a ferulic acid or ferulate ester rich solution or treatment can be used to precipitate lignin-derived or other high-polarity impurities from a ferulic acid or ferulate ester rich semi-solid, solid, or oil like residue which can optionally be a product of the previously described solvent based oil extraction step. In some embodiments, an organic solvent (solvent a) including but not limited to ethyl acetate, toluene, or acetone can be added to the ferulic acid or ferulate ester rich semi-solid, solid, or oil like residue (in 1:1 to 50:1 weight ratio) to precipitate a certain amount of fine powders. A second organic solvent (solvent b) including but not limited to heptane, toluene, petroleum ether, hexane, or a similar solvent is then added where the volume ratio of solvent b to solvent a in the mixture ranges from 1:100 to 1:1, to reduce the polarity and further precipitate impurities. The ferulic acid maintains high solubility in the mixture of solvent a and solvent b and >85 wt. % of ferulic acid can be extracted from the ferulic acid/ferulate ester rich semi-solid, solid, or oil like residue into the solution. Precipitated solids can be removed from the liquid solution by filtration methods. The filtered liquid solution can then be concentrated to dryness and the purity of ferulic acid in the concentrate can be in the range of 35-65 wt. % pure. Relevant process alternatives to solvent based precipitation include but are not limited to cation exchange, anion exchange, simulated moving bed chromatography, liquid chromatography, counter current chromatography, ultrafiltration, nanofiltration, rotating disk ultra or nano filtration, or sublimation.

Selective adsorption: In some embodiments selective adsorption is a purification process in which impurities can be removed from the ferulic acid through different affinity to the adsorbents. For example, a ferulic acid or ferulate ester containing mixture can be dissolved in a solvent where the solvent can include but is not limited to ethanol, acetone, ethyl acetate, or the mixture of at least two above mentioned solvents in mass ratio of 1:1 to 1:100. To this mixture can be added selective adsorbents which can include but are not limited to silica gel, activated carbon, clay minerals, polystyrene-divinylbenzene based resins, polystyrene-divinylbenzene based resins with amine or tertiary amine functionalization, acrylic based resins, similar materials, and the solvent to adsorbent mass ratio ranges from 1000:1 to 1:1. The mixture can be stirred at 0-60° C. for 0.1-24 hours and the adsorbent can be removed through filtration or sedimentation. The liquid solution can then be concentrated to dryness to achieve a product with higher ferulic acid or ferulate ester purity than that which was initially charged to the solution. Relevant process alternatives to selective adsorption include but are not limited to cation exchange, anion exchange, simulated moving bed chromatography, liquid chromatography, counter current chromatography, ultrafiltration, nanofiltration, rotating disk ultra or nano filtration, or sublimation. In some embodiments selective adsorption removes compounds that inhibit the biological transformation of ferulic acid to vanillin.

In some embodiments, a crude extract solution containing ferulic acid or ferulate, or any aqueous or organic process stream containing ferulic acid or ferulate can be further purified through use of ultrafiltration. Ultrafiltration can be used to selectively remove biomass extract fractions such as lignin, carbohydrates, arabinoxylans, hemicellulose and derivatives of such from ferulic acid and ferulate products.

In some embodiments, flocculation agents can be added to the crude extract solution or intermediate process streams to precipitate impurities including but not limited to lignin, carbohydrates, hemicellulose, arabinoxylans and derivatives of such from ferulic acid and ferulate containing products. Flocculation agents can include, but are not limited to cationic or anionic flocculation agents.

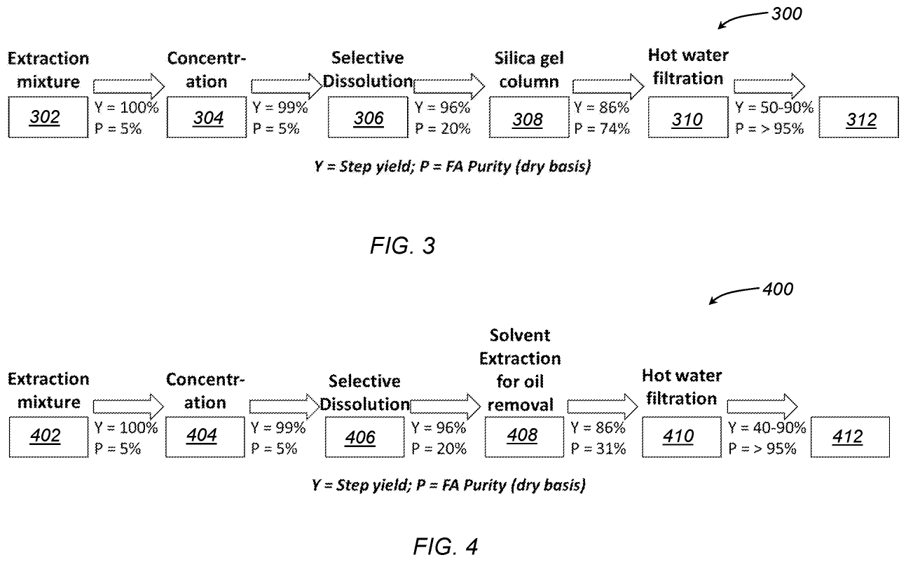

Examples of the overall purification processes are given with regard to FIGS. 3-10 below. In these examples, any of the purification processes and parameters are described herein can be used, and the descriptions with respect to the figures are not intended to be limiting.

In some embodiments, a process 300 for the purification of ferulic acid can comprise concentration of the extraction mixture from step 302 at step 304, followed by a selective dissolution step 306, a silica adsorption step 308 and a hot water filtration step 310 to produce the ferulic acid product at step 312. In some embodiments, the crude extraction mixture can comprise between about 1-10 wt. % ferulic acid. As shown in FIG. 3, the process 300 can begin with a crude extraction mixture 302 (e.g., ferulic acid rich liquid product of the reactive extraction) that can be processed in a concentration step 304 to form a concentrated mixture. For example, the crude extraction mixture can be concentrated through vacuum evaporation. In some embodiments, the concentrated mixture can comprise between about 1-10 wt. % ferulic acid, where the concentration of ferulic acid can be higher than that in the crude extraction mixture. The pH of the concentrated mixture can be adjusted to between about 3-4 by adding an acid (e.g., hydrochloric acid, etc.). The acidified mixture can be combined with an organic solvent, including any of those described herein such as ethyl acetate and under conditions as described herein. Alternatively, following partial concentration of the crude extraction mixture and acidification, the remaining liquid can be spray dried to form a powder-like solid and added to the solvent solution used for selective dissolution as step 306. After the mixing, two phases of liquid (organic and aqueous) can be allowed to separate. The organic layer can be separated using and concentrated to achieve the intermediate product. In some embodiments, the step yield of ferulic acid can be between about 90-98%, and the ferulic acid purity can be between about 10-30 wt. %, or between about 15-25 wt. %. The selective dissolution in an organic solvent such as ethyl acetate can remove water-soluble impurities.

In the next step 308, the low polarity (e.g., corn oils) and high polar (e.g., lignin-derived oligomers) impurities can be removed using the silica gel column. In this step, the intermediate product can be applied on the top of a silica gel column. The ferulic acid-rich fractions are collected and concentrated to achieve the concentrated product. The silica gel adsorption step can have an 80-90% step yield of ferulic acid, and the resulting ferulic acid purity can be between about 60-90 wt. %, between about 70-85 wt. %.

The concentrated product can be added to water in a hot water filtration step 310 and the mixture can be heated to boiling to achieve a yellowish liquid portion and a fraction of precipitate. The liquid portion is filtered when hot and left to cool to room temperature. The solution is then cooled and left to allow the ferulic acid to precipitate. When the precipitation is finished, the mixture is filtered to collect the ferulic acid product at step 312. The hot water filtration step can have a 50-90% step yield of ferulic acid, and the resulting ferulic acid product can have a ferulic acid purity of greater than or equal to 95 wt. %. The hot water filtration step can be repeated to further increase the purity to a desired level.

FIG. 4 depicts another embodiment of a purification process 400. As depicted in FIG. 4, a crude extraction mixture at step 402 (e.g., comprising ferulic acid rich liquid product of the reactive extraction) can be concentrated at step 404, for example through vacuum evaporation to form a concentrated mixture. The concentrated mixture can then be processed using a selective dissolution process at step 406. The pH of the concentrated mixture can be adjusted to 3-4 by adding an acid. The acidified mixture can be added into an organic solvent. Alternatively, following partial concentration of the crude extraction mixture and acidification, the remaining liquid can be spray dried to form a powder-like solid and added to the organic solvent used for selective dissolution at step 406. After the combining and agitating, two phases of liquid (organic and aqueous) can form. The organic layer can be separated and concentrated to achieve an intermediate product. In some embodiments, the step yield of ferulic acid can be between about 90-98%, and the ferulic acid purity can be between about 10-30 wt. %, or between about 15-25 wt. %. The selective dissolution in step 406 in an organic solvent (including any of those disclosed herein) can remove water-soluble impurities including but not limited to salts, sugars, carbohydrates, etc.

In the organic extraction step 408, the low polar impurities like corn oils can be removed through extraction with an organic solvent. The product from the dissolution step 406 can be added to an organic solvent in volume ratio of between about 1:20 to 1:1, or about 1:10. The organic solvent-rich liquid portion can be collected. The organic extraction step can be repeated for another 2-3 times if needed to achieve a desired separation. After extraction with the organic solvent, the resulting product can be turned into a highly viscous semi-solid. In some embodiments, the step yield of ferulic acid can be between about 75-95% or between 80-90%, and the ferulic acid purity can be between about 20-40 wt. %, or between about 25-35 wt. %.

Hot water filtration can then be used in step 410 to further purify the product of the organic extraction from step 408. The product can be added to water and the mixture is heated to boiling to achieve a yellowish liquid layer and a fraction of dark oily layer. The yellowish liquid portion can be filtered when hot and left to cool to room temperature. The solution is then left for ferulic acid to precipitate. When the precipitation is finished, the mixture can be filtered to collect the product at step 412. In some embodiments, the step yield of ferulic acid can be between about 40-90%, and the ferulic acid purity can be greater than 90 wt. %. The hot water filtration step 410 can be repeated to increase the ferulic acid purity to be >95%.

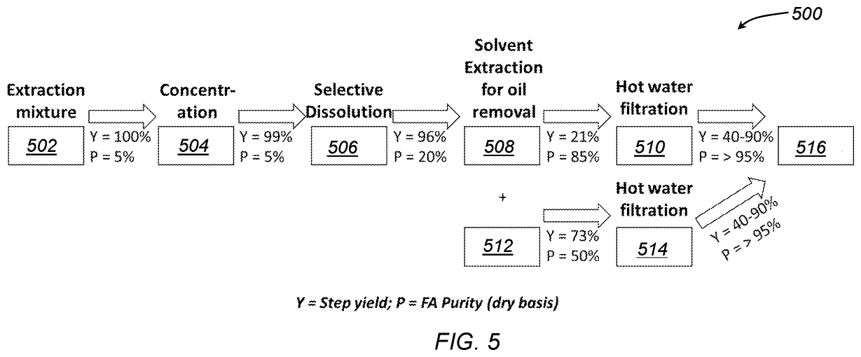

FIG. 5 illustrates another embodiment of a purification process 500. As depicted in FIG. 5, the crude extraction mixture from step 502 (e.g., a ferulic acid rich liquid product of the reactive extraction) can be concentrated in step 504. In some aspects, the mixture can be concentrated through vacuum evaporation. The pH of the concentrated mixture from step 504 can be adjusted to 3-4 by adding an acid. The acidified solution can be added into an organic solvent such as ethyl acetate. Alternatively, following partial concentration of the crude extraction mixture and acidification, the remaining liquid can be spray dried to form a powder-like solid and added to the organic solution used for selective dissolution at step 506. After the stirring, two phases of liquid (organic and aqueous) can be formed. The organic layer can be separated using a separatory funnel and concentrated to achieve an intermediate product. In some embodiments, the selective dissolution step 506 can have a step yield of ferulic acid can be between about 90-98%, and the ferulic acid purity can be between about 10-30 wt. %, or between about 15-25 wt. %. The selective dissolution in ethyl acetate can remove water-soluble impurities.

In the next step 508, the low polar impurities like corn oils can be removed through extraction with an organic solvent such as hexane. The intermediate product from step 506, in which the volatile content can be below 15 wt. %, can be added to a stirred organic solvent in volume ratio of between about 1:20 to about 1:1, or about 1:10. Three fractions can result from the contact with the organic solvent: a yellowish liquid (hexane rich layer), a light brown fine powder, and a dark brown viscous semi-solid. The light brown fine powder can move freely, and it is filtered and dried to a fine powder that can be used in step 510. The ferulic acid recovery in step 508 can be between about 15-25%, and the ferulic acid purity of can be between about 75-90 wt. % or about 80-85 wt. %. After the yellowish liquid (the organic solvent rich layer) and fine powder are removed, the dark brown viscous semi-solid can be contacted with a polar solvent (e.g., acetone) and stirred in step 512. A layer of fine precipitate can form. To this mixture is added another organic solvent such as hexane to precipitate more solids. The precipitates can be removed through filtration and the solvent solution can be concentrated to achieve an additional product from step 512. The step yield of ferulic acid can be between about 60-80% or between about 70-75%, and the ferulic acid purity can be between about 40-60 wt. % or between about 45-55 wt. %. Both products can be further purified to produce a final ferulic acid product 516 using the hot water filtration process 510, 514 as described herein to achieve 40-90% step yield and >90% ferulic acid purity. The hot water filtration step can be repeated to boost the ferulic acid purity to >95%.

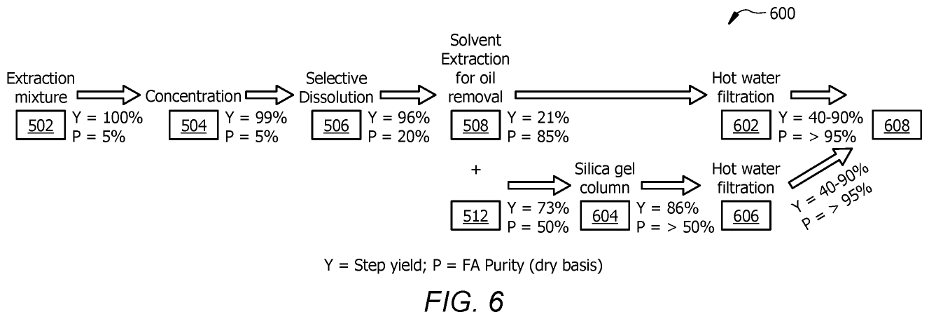

FIG. 6 illustrates another purification process. As shown in FIG. 6, the first steps using an extraction mixture at step 502, concentrating the mixture at step 504, performing selective dissolution and using an organic extraction process at steps 508 and 512 to produce three fractions can be the same as or similar to these steps as described with respect to FIG. 5. In the process 600, the product from the organic extraction process at step 512 can be applied on the top of a silica gel column and the column can be flushed with one or more organic solvents. The ferulic acid-rich fractions can be collected and concentrated to achieve a product used in a hot water filtration process. Both products from steps 508 and 604 can be purified using the hot water filtration in steps 602, 606 to produce a ferulic acid product 608, which can be the same as or similar to those described with respect to FIG. 5 in steps 510, 514. The hot water filtration step can be repeated as desired to boost the ferulic acid purity to >95%.

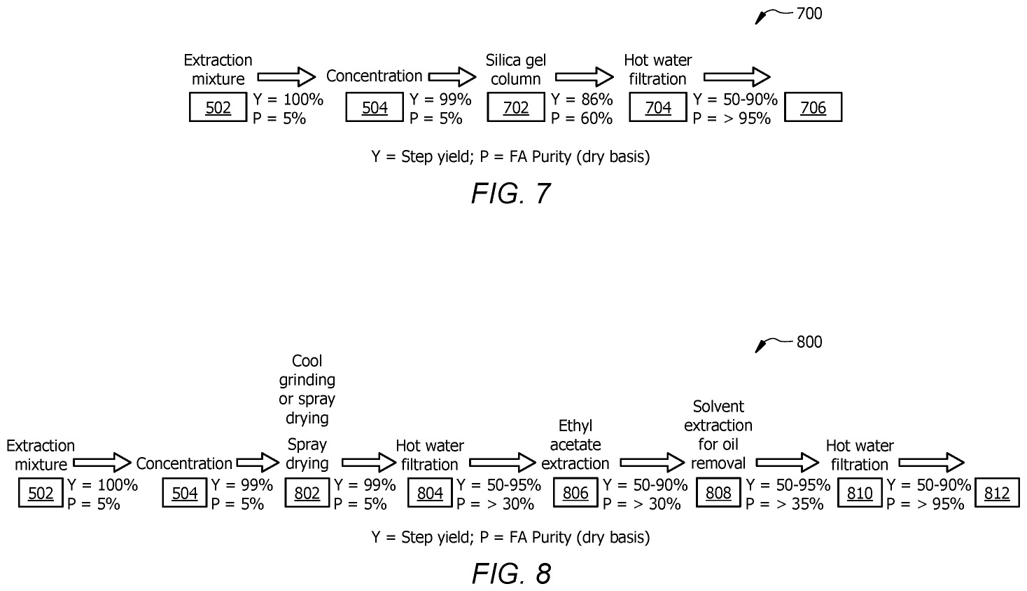

Another purification process 700 is depicted in FIG. 7. As shown in FIG. 7, the first steps using an extraction mixture at step 502 and concentrating the mixture at step 504 can be the same as or similar to these steps as described with respect to FIG. 5. The resulting product mixture from the concentration step 504 can be passed to a silica gel column in step 702. The mixture can be applied on the top of a silica gel column and the column can be flushed one or more organic solvents. The organic solvents can comprise polar or non-polar organic solvents. The ferulic acid-rich fractions from the column can be collected and concentrated to achieve an intermediate product. The gel column purification step 702 can have a step yield of between about 75-95%, or between about 80-90%, and the ferulic acid can have a purity of between about 50-70 wt. %, or between about 55-65 wt. %. The resulting product can be purified using the hot water filtration in step 704, which can be the same as or similar to those described with respect to FIG. 5 in steps 510. The hot water filtration step can be repeated as desired to boost the ferulic acid purity in the ferulic acid product 706 to >95%.

Another purification process 800 is shown in FIG. 8. As shown in FIG. 7, the first steps using an extraction mixture at step 502 and concentrating the mixture at step 504 can be the same as or similar to these steps as described with respect to FIG. 5. The resulting concentrated solution from the concentration step 504 can be mixed with liquid nitrogen or dry ice to turn it into a solid state in a cool grinding process in step 802. The solid can be ground into powder at temperature below 10° C. Alternatively, following concentration of the crude extraction mixture to the concentrated solution, the liquid can be spray dried to form a powder-like solid. The powder from cool grinding or spray drying can be passed to a hot water filtration step that can be the same or similar to those described herein (e.g., with respect to step 510 described with respect to FIG. 5). In this process 804, solid can be added to water and the mixture can be heated to boiling to achieve a yellowish liquid portion, a dark oily portion, and a portion of precipitate. The liquid portion can be filtered when hot and left to cool to room temperature. The solution can then be left for ferulic acid to precipitate. When the precipitation is finished, the mixture can be filtered to collect the product. The step yield of ferulic acid from the hot water filtration step 804 can be between about 50-95%, and the ferulic acid purity of the product can be between about 20-40 wt. %, or between about 25-35 wt. %.

The resulting product can be passed to one or more organic extraction processes 806, 808. In some embodiments, a first organic extraction step 806 can include mixing the product with an organic solvent such as ethyl acetate to extract the ferulic acid product while the water-soluble impurities precipitate. The organic fraction can be concentrated to an intermediate product. The step yield of ferulic acid from the organic extraction step 806 can be between about 50-95%, and the resulting ferulic acid purity can be between about 20-40 wt. %. The intermediate product can be combined with a second organic solvent, which can be different than the organic solvent used in step 806, to remove the corn oils and achieve a product from step 808. The step yield of ferulic acid from step 808 can be between about 50-95%, and the ferulic acid purity can be between about 30-40 wt. %, where the purity can be higher than the product from the extraction step 806. The resulting product from step 808 can be passed to a hot water filtration step 810, which can be the same or similar to the hot water filtration steps described herein. The hot water filtration step can be repeated as desired to boost the ferulic acid purity in the ferulic acid product 812 to >95%.

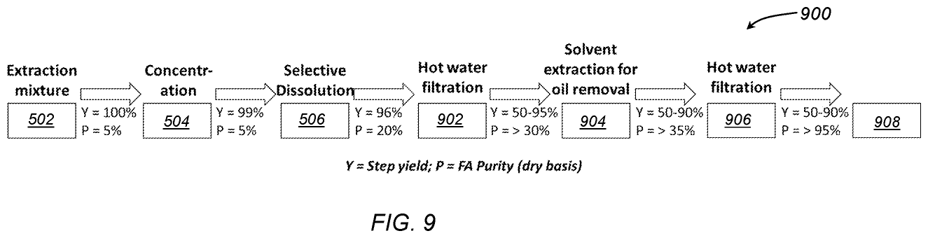

Another embodiment of a purification process 900 is shown in FIG. 9. As shown in FIG. 9, the first steps using an extraction mixture at step 502, concentrating the mixture at step 504 and using a selective dissolution process in step 506 can be the same as or similar to these steps as described with respect to FIG. 5. The product from the selective dissolution step 506 can be passed to a hot water filtration process 902. In this step, the product can be added to water and the mixture can be heated to boiling to achieve a yellowish liquid portion, a dark oily portion, and a portion of precipitate. The liquid portion can be filtered when hot and left to cool to room temperature. The solution can then be left for ferulic acid to precipitate. When the precipitation is finished, the mixture can be filtered to collect the intermediate product. The hot water filtration process can have a step yield of ferulic acid of 50-95%, and the ferulic acid purity of the product from step 902 can be greater than about 30 wt. %. The product can then pass to an organic extraction step 904. In this process, the intermediate product can be extracted with an organic solvent to remove the corn oils and achieve a product. The organic extraction process 904 can have a step yield of ferulic acid of 50-95%, and the ferulic acid purity of the product from step 902 can be greater than about 35 wt. %. The product 5 can then be passed to a hot water filtration step 906 to further purify the ferulic acid. The hot water filtration step 906 can be the same as or similar to any of the hot water filtration steps described herein. The hot water filtration step can be repeated as desired to boost the ferulic acid purity in the ferulic acid product 908 to >95%.

### EXAMPLES

The subject matter having been generally described, the following examples are given as particular aspects of the disclosure and are included to demonstrate the practice and advantages thereof, as well as preferred aspects and features of the inventions. It should be appreciated by those of skill in the art that the techniques disclosed in the examples which follow represent techniques discovered by the inventors to function well in the practice of the inventions, and thus can be considered to constitute preferred modes for its practice. However, those of skill in the art should, in light of the present disclosure, appreciate that many changes can be made in the specific aspects which are disclosed and still obtain a like or similar result without departing from the scope of the inventions of the instant disclosure. It is understood that the examples are given by way of illustration and are not intended to limit the specification of the claims to follow in any manner.

### Example 1.A-F

Reactive Extraction of Ferulic Acid and Ethyl Ferulate in Ethanol/Water. In this example, 350 g of corn on a dry mass basis is used. For this example, variances in wet mass of corn fiber, corn moisture content, ethanol percent by weight (wt. %) in the solvent, water added, ethanol added, sodium hydroxide concentration, and sodium hydroxide added are tabulated. With these tabulated variables, expected percent yields of tracked extractives are also tabulated. To start, the wet mass of corn fiber is weighed based on the dry mass of said fiber and is loaded into a 4 L stirred glass batch reactor. The amount of water and ethanol to be added to the system is computed by defining a solvent to dry biomass ratio of 6.3:1, and defining the wt. % of ethanol in water. Part of the water already exists in the corn fiber moisture content. The remaining amount of water is measured. Sodium hydroxide (NaOH) concentration is determined on a moles per liter of total solvent basis. The computed amount of NaOH is weighed and dissolved in the water. This water, along with the required amount of ethanol to make the necessary wt. % ethanol in the solvent system, were added to the reactor. The reactor was closed, set in a hot water bath held at 80° C., equipped with a reflux condenser to prevent pressure buildup and/or solvent loss, stirred with the impeller motor set to 400 rpm. The reactor temperature rises to 80° C. over the course of 1 hour. The reactor continues to heat and stir for another hour (2 hours total reactor time). The solids are then filtered in a 75 μm filter bag. The filtered solids are rinsed with 2.0 L of wt. % ethanol twice (4 L total). Fine solids were removed from the combined extract by filtering through 11 μm filter paper. The yields of ferulic acid and ethyl ferulate was determined by HPLC. The ferulic acid and ethyl ferulate yields are computed from the chromatogram with respect to the 350 g of dry corn fiber (403 g wet corn fiber at 13% moisture).

At this point, the dissolved solids in the crude extract is approximately 5% ferulic acid. To increase the purity of the crude extract, a selective dissolution is performed. This is accomplished by concentrating the extract to a viscous sludge and dissolving the sludge in ethyl acetate. 88% of the total extracted ferulic acid is recovered in the selectively dissolved solids in the ethyl acetate phase, which is approximately 20% ferulic acid. The remaining 80% of the dissolved solids are a mixture mainly comprised of corn oils and phenolic lignin derivatives.

TABLE 1

| | Corn Fiber | Corn | Solvent | Water | Ethanol | NaOH | NaOH |
|---|---|---|---|---|---|---|---|
| Example | Wet Mass [g] | Moisture | Ethanol wt % | Added [g] | Added [g] | Conc. [M] | Added [g] |
| 1.a | 403 | 13% | 70% | 609 | 1544 | 0.5 | 52.35 |
| 1.b | 403 | 13% | 70% | 609 | 1544 | 0.3 | 31.41 |
| 1.c | 875 | 60% | 70% | 137 | 1544 | 0.5 | 52.35 |
| 1.d | 875 | 60% | 70% | 137 | 1544 | 0.3 | 31.41 |
| 1.e | 403 | 13% | 80% | 389 | 1764 | 0.5 | 53.52 |
| 1.f | 403 | 13% | 80% | 389 | 1764 | 0.3 | 32.11 |

| | Yields as a percent of the original dry mass of corn | | | | | | | |
|---|---|---|---|---|---|---|---|---|
| | | | Total | | | | | |
| | Ferulic | Ethyl | Ferulate | Oleic | Linoleic | Ethyl | Ethyl | Total Oils |
| Example | Acid | Ferulate | Yield | Acid | Acid | Oleate | Linoleate | Yield |
| 1.a | 1.8% | 0.1% | 1.9% | 0.70% | 1.7% | — | — | 2.4% |
| 1.b | 1.2% | 0.5% | 1.7% | 0.7% | 1.3% | — | — | 2.0% |
| 1.c | 1.5% | 0.4% | 1.8% | 0.2% | 0.5% | — | 0.1% | 0.8% |
| 1.d | 1.3% | 0.6% | 1.9% | 0.3% | 0.7% | | 0.1% | 1.1% |
| 1.e | 1.6% | 0.1% | 1.7% | 0.7% | 1.5% | 0.2% | — | 2.4% |
| 1.f | 0.7% | 0.5% | 1.2% | 0.7% | 1.3% | — | 0.1% | 2.1% |

### Example 2

Reactive Extraction of Ferulic Acid in Water. In this example, 375 g of corn fiber that is 60% by weight water was loaded into a 4 L stirred glass batch reactor. In 2.78 L of water, 36 g of sodium hydroxide was dissolved, and the solution was added to the reactor. This liquid, along with the moisture content in the corn fiber yields a 20:1 water to dry biomass ratio, with an overall sodium hydroxide concentration of 0.3 M. The reactor was closed, set in a hot water bath held at 30° C., equipped with a reflux condenser to prevent pressure buildup and/or solvent loss, stirred with the impeller motor set to 200 rpm. The reactor temperature rises to 30° C. over the course of 1 hour. The reactor continues to heat and stir for another 2 hours (3 hours total reactor time). The solids are then filtered in a 75 μm filter bag. The filtered solids are rinsed with 1.5 L of water twice (3 L total). The yield of ferulic acid was determined by HPLC. The ferulic acid yield was determined to be 2.1% with respect to the 150 g of dry corn fiber (375 g wet corn fiber at 60% moisture).

Using this extraction method, phenolic lignin derivatives are not extracted; however, arabinoxylans will be extracted from the hemicellulose. The intent of not using ethanol in this example is to avoid the concentration step that precedes the selective dissolution in example 1, directly moving to a liquid-liquid extraction with ethyl acetate to purify the ferulic acid. However, arabinoxylans, also known as corn fiber gum, is a known emulsifier, and when the extract is mixed with ethyl acetate, a single emulsified phase is formed. The arabinoxylans are removed from the system via basic hydrolysis at 0.5M for 3 hrs. By hydrolyzing the arabinoxylans to arabinose and xylose sugars, the extract can be filtered. The resulting extract is then put through a 2-phase liquid-liquid extraction with ethyl acetate. 98% of the ferulic acid is recovered in this extraction, and the dissolved solids in the ethyl acetate extract is 33% ferulic acid.

### Example 3

Reactive Extraction of Ferulic Acid in Water with Ethanol Rinse. In this example, 403 g of corn fiber that is 13% by weight water was loaded into a 4 L stirred glass batch reactor. In 2.75 L of water, 33 g of sodium hydroxide was dissolved, and the solution was added to the reactor. This liquid, along with the moisture content in the corn fiber yields a 8:1 water to dry biomass ratio, with an overall sodium hydroxide concentration of 0.3 M. The reactor was closed, set in a hot water bath held at 30° C., equipped with a reflux condenser to prevent pressure buildup and/or solvent loss, stirred with the impeller motor set to 400 rpm. The reactor temperature rises to 30° C. over the course of 1 hour. The reactor continues to heat and stir for another 2 hours (3 hours total reactor time). The reactor slurry is then diluted with 90 wt. % ethanol until the total solvent concentration of ethanol is 40 wt. % (2 L). The solids are then filtered in a 75 μm filter bag. The filtered solids are rinsed with 1 L of 90 wt. % ethanol twice (2 L total). Fine solids were removed from the combined extract by filtering through 11 μm filter paper. The yield of ferulic acid was determined by HPLC. The ferulic acid yield was determined to be 2.1% with respect to the 350 g of dry corn fiber (403 g wet corn fiber at 13% moisture).

Using this extraction method, phenolic lignin derivatives are not extracted. Although arabinoxylans will be extracted from the hemicellulose, arabinoxylans are insoluble in ethanol/water mixtures above 40 wt. % ethanol, hence the addition of ethanol to the crude extract before filtering the solids. Since ethanol has been introduced to the system, a selective dissolution is performed. This is accomplished by concentrating the extract to a viscous sludge and dissolving the sludge in ethyl acetate. 88% of the total extracted ferulic acid is recovered in the selectively dissolved solids in the ethyl acetate phase, which is approximately 40% ferulic acid by mass when the solvent is removed. This is a two-fold improvement compared to the ethanol/water extraction.

### Example 4

Following the scheme depicted in FIG. 3: 2 L of the crude extraction mixture 1 (ferulic acid rich liquid product of the reactive extraction) is concentrated to 2 (180 mL) through vacuum evaporation at 55° C. The pH of the concentrated mixture 2 is adjusted to 3-4 by adding hydrochloric acid. The acidified 2 is slowly added into 540 mL of ethyl acetate when stirred and the mixture is agitated at 25° C. for 3 hours. Alternatively, following partial concentration of the crude extraction mixture 1 to ca. 180 mL and acidification, the remaining liquid can be spray dried to form a powder-like solid and added to the ethyl acetate solution used for selective dissolution 3. After the mixing, two phases of liquid (organic and aqueous) are observed. The organic layer is separated using a separatory funnel and concentrated to achieve the product 3 (96% step yield of ferulic acid, 20% ferulic acid purity). The selective dissolution in ethyl acetate can remove water-soluble impurities. In the next step, the low polarity (e.g., corn oils) and high polar (e.g., lignin-derived oligomers) impurities are removed using the silica gel column. Product 3 is applied on the top of a silica gel column (silica gel weight is ˜ 40 g) and the column is flushed with the mixture of acetone/hexane (v/v ranges from 0:1 to 1:1). The ferulic acid-rich fractions are collected and concentrated to achieve the product 4 (86% step yield of ferulic acid, 74% ferulic acid purity). Product 4 is added to 15 mL of water (pH=6) and the mixture is heated to boiling to achieve a yellowish liquid portion and a fraction of precipitate. The liquid portion is filtered when hot and left to cool to room temperature. The solution is then left at 4° C. for 6 hours for ferulic acid to precipitate. When the precipitation is finished, the mixture is filtered to collect the product 5 (50-90% step yield of ferulic acid, >95% ferulic acid purity). The hot water filtration step can be repeated to further increase the purity if needed.

### Example 5

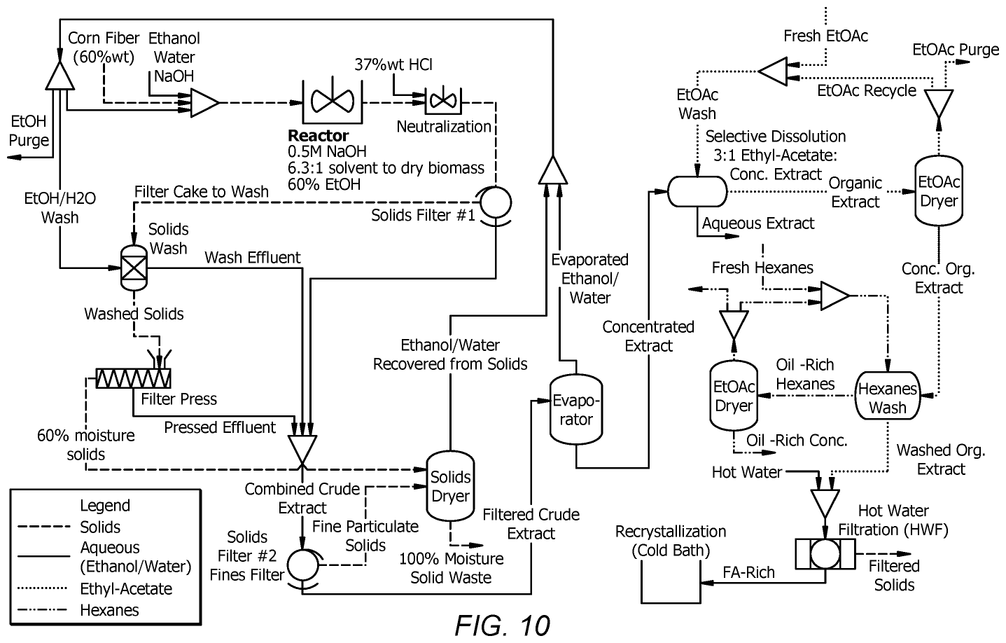

As depicted in FIG. 4, 2 L of the crude extraction mixture 1 (ferulic acid rich liquid product of the reactive extraction) is concentrated to 2 (180 mL) through vacuum evaporation at 55° C. The pH of the concentrated mixture 2 is adjusted to 3-4 by adding hydrochloric acid. The acidified 2 is slowly added into 540 mL of ethyl acetate when stirred and the mixture is agitated at 25° C. for 3 hours. Alternatively, following partial concentration of the crude extraction mixture 1 to ca. 180 mL and acidification, the remaining liquid can be spray dried to form a powder-like solid and added to the ethyl acetate solution used for selective dissolution 3. After the stirring, two phases of liquid (organic and aqueous) are observed. The organic layer is separated using a separatory funnel and concentrated to achieve the product 3 (96% step yield of ferulic acid, 20% ferulic acid purity). The selective dissolution in ethyl acetate can remove water-soluble impurities including but not limited to salts, sugars, carbohydrates, etc. In the next step, the low polar impurities like corn oils are removed through extraction with hexane. Product 3 is slowly added to a stirred hexane solvent in 1:10 volume ratio. The mixture is fast stirred at 25° C. for 0.5 hours. The hexane-rich liquid portion is collected. Repeat the hexane extraction step for another 2-3 times if needed. After hexane extraction, the product 3 (originally dark oily liquid) is turned into a highly viscous semi-solid (product 4, 86% process yield of ferulic acid, 31% ferulic acid purity, no corn oils observed). Product 4 is added to 15 mL of water (pH=6) and the mixture is heated to boiling to achieve a yellowish liquid layer and a fraction of dark oily layer. The yellowish liquid portion is filtered when hot and left to cool to room temperature. The solution is then left at 4° C. for 6 hours for ferulic acid to precipitate. When the precipitation is finished, the mixture is filtered to collect the product 5 (40-90% step yield of ferulic acid, >90% ferulic acid purity). The hot water filtration step is repeated once to boost the ferulic acid purity to be >95%. An example of an extraction process followed by the purification as described in this example is provided in FIG. 10.

### Example 6

As depicted in FIG. 5, 2 L of the crude extraction mixture 1 (ferulic acid rich liquid product of the reactive extraction) is concentrated to 2 (180 mL) through vacuum evaporation at 55° C. The pH of the concentrated mixture 2 is adjusted to 3-4 by adding hydrochloric acid. The acidified 2 is slowly added into 540 mL of ethyl acetate when stirred and the mixture is agitated at 25° C. for 3 hours. Alternatively, following partial concentration of the crude extraction mixture 1 to ca. 180 mL and acidification, the remaining liquid can be spray dried to form a powder-like solid and added to the ethyl acetate solution used for selective dissolution 3. After the stirring, two phases of liquid (organic and aqueous) are observed. The organic layer is separated using a separatory funnel and concentrated to achieve the product 3 (96% step yield of ferulic acid, 20% ferulic acid purity). The selective dissolution in ethyl acetate can remove water-soluble impurities. In the next step, the low polar impurities like corn oils are removed through extraction with hexane. Product 3, in which the volatile content is below 15 wt. %, is slowly added to a stirred hexane solvent in 1:10 volume ratio. The mixture is quickly stirred at 25° C. for 0.5 hours. Three fractions are achieved: yellowish liquid (hexane rich layer), light brown fine powder and dark brown viscous semi-solid. The light brown fine powder can move freely, and it is filtered and dried to a fine powder (product 4) with the ferulic acid recovery of 21% and ferulic acid purity of 85%. After the yellowish liquid (hexane rich layer) and fine powder are removed, the dark brown viscous semi-solid is contacted with 10 mL of acetone and the mixture is stirred at 25° C. for 10 min. A layer of fine precipitate is observed. To this mixture is added another 5 mL of hexane to precipitate more solids. The precipitates are removed through filtration and the hexane/acetone solution is concentrated to achieve product 5. (73% step yield of ferulic acid, 50% ferulic acid purity). Both product 4 and product 5 are purified using the hot water filtration process as described in Example 2 to achieve 40-90% step yield and >90% ferulic acid purity. The hot water filtration step is repeated to boost the ferulic acid purity to >95%.

### Example 7

As depicted in FIG. 6, 2 L of the crude extraction mixture 1 is concentrated to 2 (180 mL) through vacuum evaporation at 55° C. The pH of the concentrated mixture 2 is adjusted to 3-4 by adding hydrochloric acid. The acidified 2 is slowly added into 540 mL of ethyl acetate when stirred and the mixture is agitated at 25° C. for 3 hours. Alternatively, following partial concentration of the crude extraction mixture 1 to ca. 180 mL and acidification, the remaining liquid can be spray dried to form a powder-like solid and added to the ethyl acetate solution used for selective dissolution 3. After the stirring, two phases of liquid (organic and aqueous) are observed. The organic layer is separated using a separatory funnel and concentrated to achieve the product 3 (96% step yield, 20% ferulic acid purity). The selective dissolution in ethyl acetate can remove water-soluble impurities. In the next step, the low polar impurities like corn oils are removed through extraction with hexane. Product 3, in which the volatile content is below 15 wt. %, is slowly added to a stirred hexane solvent in 1:10 volume ratio. The mixture is quickly stirred at 25° C. for 0.5 hours. Three fractions are achieved: yellowish liquid (hexane rich layer), light brown fine powder and dark brown viscous semi-solid. The light brown fine powder can move freely, and it is filtered and dried to a fine powder (product 4) with the ferulic acid recovery of 21% and ferulic acid purity of 85%. After the yellowish liquid (hexane rich layer) and fine powder are removed, the dark brown viscous semi-solid is contacted with 10 mL of acetone and the mixture is stirred at 25° C. for 10 min. A layer of fine precipitate is observed. To this mixture is added another 5 mL of hexane to precipitate more solids. The precipitates are removed through filtration and the hexane/acetone solution is concentrated to achieve product 5. (73% step yield, 50% ferulic acid purity). Product 5 is applied on the top of a short silica gel column (silica gel weight is ˜ 20 g) and the column is flushed with the mixture of acetone/hexane (v/v=1:1). The ferulic acid-rich fractions are collected and concentrated to achieve the product 6 (86% step yield, >50% ferulic acid purity). Both product 4 and product 6 are purified using the hot water filtration as described in Example 2 to achieve 40-90% step yield and >90% ferulic acid purity. The hot water filtration step is repeated to boost the ferulic acid purity to >95%.

### Example 8

As depicted in FIG. 7, 2 L of the crude extraction mixture 1 (ferulic acid rich liquid product of the reactive extraction) is concentrated to 2 (30 mL) through vacuum evaporation at 55° C. Alternatively, following partial concentration of the crude extraction mixture 1, the remaining liquid can be spray dried to form a powder-like solid (Product 2) and applied to the silica gel column. Product 2 is applied on the top of a silica gel column (silica gel weight is >100 g) and the column is flushed with the mixture of acetone/hexane (v/v ranges from 0:1 to 1:1). The ferulic acid-rich fractions are collected and concentrated to achieve the product 3 (86% step yield of ferulic acid, 60% ferulic acid purity). Product 3 is added to 15 mL of water (pH=6) and the mixture is heated to boiling to achieve a yellowish liquid portion and a fraction of precipitate. The liquid portion is filtered when hot and left to cool to room temperature. The solution is then left at 4° C. for 6 hours for ferulic acid to precipitate. When the precipitation is finished, the mixture is filtered to collect the product 4 (50-90% step yield of ferulic acid, >90% ferulic acid purity). The hot water filtration step is repeated to boost the ferulic acid purity to >95%.

### Example 9

As depicted in FIG. 8, 2 L of the crude extraction mixture 1 (ferulic acid rich liquid product of the reactive extraction) is concentrated to 2 (close to dryness) through vacuum evaporation at 55° C. Product 2 is mixed with liquid nitrogen or dry ice to turn it into a solid state. The solid is grinded into powder (product 3) at temperature below 10° C. Alternatively, following concentration of the crude extraction mixture to 2 (close to dryness), the liquid can be spray dried to form a powder-like solid. The powder from cool grinding or spray drying is added to 50 mL of water (pH=6) and the mixture is quickly heated to boiling to achieve a yellowish liquid portion, a dark oily portion, and a portion of precipitate. The liquid portion is filtered when hot and left to cool to room temperature. The solution is then left at 4° C. for 6 hours for ferulic acid to precipitate. When the precipitation is finished, the mixture is filtered to collect the product 4 (50-95% step yield of ferulic acid, >30% ferulic acid purity). 20 mL of ethyl acetate is added to product 4 to extract the ferulic acid product while the water-soluble impurities precipitate. The ethyl acetate fraction is concentrated to product 5 (50-95% step yield of ferulic acid, >30% ferulic acid purity), which is extracted with 10 mL of hexane to remove the corn oils and achieve product 6 (50-95% step yield of ferulic acid, >35% ferulic acid purity). Product 6 is added to 15 mL of water (pH=6) and the mixture is heated to boiling to achieve a yellowish liquid portion and a fraction of precipitate. The liquid portion is filtered when hot and left to cool to room temperature. The solution is then left at 4° C. for 6 hours for ferulic acid to precipitate. When the precipitation is finished, the mixture is filtered to collect the product 7 (50-90% step yield of ferulic acid, >80% ferulic acid purity). The hot water filtration step is repeated to boost the ferulic acid purity to >95%.

### Example 10

As depicted in FIG. 9, 2 L of the crude extraction mixture 1 (ferulic acid rich liquid product of the reactive extraction) is concentrated to 2 (180 mL) through vacuum evaporation at 55° C. The pH of the concentrated mixture 2 is adjusted to 3-4 by adding hydrochloric acid. The acidified 2 is slowly added into 540 mL of ethyl acetate when stirred and the mixture is agitated at 25° C. for 3 hours. After the stirring, two phases of liquid (organic and aqueous) are observed. The organic layer is separated using a separatory funnel and concentrated to achieve the product 3 (96% step yield, 20% ferulic acid purity). Alternatively, following partial concentration of the crude extraction mixture 1, the remaining liquid can be spray dried to form a powder-like solid and added to selective dissolution step 3. The selective dissolution in ethyl acetate can remove water-soluble impurities from the organic phase containing the ferulic acid product. Product 3 is added to 30 mL of water (pH=6) and the mixture is quickly heated to boiling to achieve a yellowish liquid portion, a dark oily portion, and a portion of precipitate. The liquid portion is filtered when hot and left to cool to room temperature. The solution is then left at 4° C. for 6 hours for ferulic acid to precipitate. When the precipitation is finished, the mixture is filtered to collect the product 4 (50-95% step yield, >30% ferulic acid purity). Product 4 is extracted with 10 mL of hexane to remove the corn oils and achieve product 5 (50-95% step yield, >35% ferulic acid purity). Product 5 is added to 15 mL of water (pH=6) and the mixture is heated to boiling to achieve a yellowish liquid portion and a fraction of precipitate. The liquid portion is filtered when hot and left to cool to room temperature. The solution is then left at 4° C. for 6 hours for ferulic acid to precipitate. When the precipitation is finished, the mixture is filtered to collect the product 6 (50-90% step yield, >80% ferulic acid purity). The hot water filtration step is repeated to boost the ferulic acid purity to >95%.

### Example 11

Conversion of 90% pure ferulic acid to vanillin using ferulic acid purified in Example 10. In this example, a seed culture is prepared by adding Amycolatopsis sp. ATCC39116 cells from an agar plate or from cryopreserved cell samples into a 250 mL baffled Erlenmeyer flask containing 30 mL sterile fermentation broth consisting of 4 g/L Yeast extract; 0.2 g/L NaCl; 0.05 g/L CaCl2 ·2H2O; 0.2 g/L MgSO4·7H2O; 4 g/L Na2HPO4, 1 g/L KH2PO4, 10 g/L Dextrose. The cultures are placed in a shaking incubator for 18 hours (+28° C. at 180 rpm). The seed culture is subsequently used to inoculate 30 mL production cultures at 4% v/v. The production cultures are placed in the shaking incubator for approximately 48 hours or until the culture reaches an OD600 15-20, at which time bioconversion is started by addition of Ferulic acid at a final concentration of 6 g/L. Prior to addition, the ferulic acid is dissolved in 1M NaOH and filtered through a 0.22 μm syringe filter. The cultures are further incubated at +28° C. at 180 rpm and samples for analysis are taken after 16-36 hours. The conversion of ferulic acid and formation of metabolites was analyzed by HPLC. HPLC analysis showed that a maximum of 58% molar yield vanillin was reached after 20 hours of induction using a sample of 90% purity.

### Example 12

Conversion of 99% pure commercially sourced ferulic acid to vanillin. In this example, a seed culture is prepared by adding Amycolatopsis sp. ATCC39116 cells from an agar plate or from cryopreserved cell samples into a 250 mL baffled Erlenmeyer flask containing 30 mL sterile fermentation broth consisting of 4 g/L Yeast extract; 0.2 g/L NaCl; 0.05 g/L CaCl2·2H2O; 0.2 g/L MgSO4·7H2O; 4 g/L Na2HPO4, 1 g/L KH2PO4, 10 g/L Dextrose. The cultures are placed in a shaking incubator for 18 hours (+28° C. at 180 rpm). The seed culture is subsequently used to inoculate 30 mL production cultures at 4% v/v. The production cultures are placed in the shaking incubator for approximately 48 hours or until the culture reaches an OD600 15-20, at which time bioconversion is started by addition of commercially available pure ferulic acid at a final concentration of 6 g/L (CAS: 1135-24-6), prior to addition, the ferulic acid is dissolved in 1M NaOH and filtered through a 0.22 μm syringe filter. The cultures are further incubated at +28° C. at 180 rpm and samples for analysis are taken after 16-36 hours. The conversion of ferulic acid and formation of metabolites was analyzed by HPLC. The conversion of ferulic acid and formation of metabolites was analyzed by HPLC. HPLC analysis showed that a maximum of 58% molar yield vanillin was reached after 20 hours of induction using commercially sourced ferulic acid of 90% purity.

### Example 13

In a prophetic example, the ferulic acid products of examples 1-10 are converted through biological means to produce a final product of vanillin. The conversion of ferulic acid to vanillin is enabled by biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and it is noted that those skilled in the art will be aware of other microorganisms and biological agents that can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system.

### Example 14

In a prophetic example, the ferulic acid intermediate products from examples 1-10, each with a purity of less than 95% pure ferulic acid are converted through biological means to produce a final product of vanillin. The conversion of ferulic acid to vanillin is enabled by biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and it is noted that those skilled in the art will be aware of other microorganisms and biological agents that can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system.

### Example 15

In a prophetic example the ethyl acetate solvent is removed from the ferulic acid products of example 1 a-f and the ferulic acid products were converted through biological means to produce a final product of vanillin. The conversion of ferulic acid to vanillin is enabled by biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and it is noted that those skilled in the art will be aware of other microorganisms and biological agents that can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system.

### Example 16

In a prophetic example, the ethyl acetate solvent was removed from the about 33% pure ferulic acid product. The resulting ferulic acid was then converted through biological means to produce a final product of vanillin. The conversion of ferulic acid to vanillin is enabled by biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and it is noted that those skilled in the art will be aware of other microorganisms and biological agents that can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system.

### Example 17

In a prophetic example, the ethyl acetate solvent was removed from the selective dissolution product of example 3, yielding ferulic acid with a purity of about 40%. The resulting ferulic acid was then converted through biological means to produce a final product of vanillin. The conversion of ferulic acid to vanillin is enabled by biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and it is noted that those skilled in the art will be aware of other microorganisms and biological agents that can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system.

### Example 18

In a prophetic example, ferulic acid of ≤90% purity is converted through biological means to produce a final product of vanillin. The vanillin product is contained in a unique product mixture where impurities from the initial ferulic acid are found in the vanillin product mixture. The conversion of ferulic acid to vanillin is enabled by biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and it is noted that those skilled in the art will be aware of other microorganisms and biological agents that can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system. The ≤90% pure ferulic acid used for this example contains ≥10% lignin or lignin derived compounds as contaminants. The product mixture contains 22.5-90% vanillin; 0-67.5% other bioproducts including but not limited to guaiacol, vanillic acid, vanillyl alcohol, 4-vinylguaiacol, protocatechuic acid, catechol, and ferulic acid; and 0-10% lignin or lignin derived compounds.

### Example 19

In a prophetic example, ferulic acid of ≤80% purity is converted through biological means to produce a final product of vanillin. The vanillin product is contained in a unique product mixture where impurities from the initial ferulic acid are found in the vanillin product mixture. The conversion of ferulic acid to vanillin is enabled by biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and it is noted that those skilled in the art will be aware of other microorganisms and biological agents that can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system. The ≤80% pure ferulic acid used for this example contains ≥20% lignin or lignin derived compounds as contaminants. The product mixture contains 20-80% vanillin; 0-60% other bioproducts including but not limited to guaiacol, vanillic acid, vanillyl alcohol, 4-vinylguaiacol, protocatechuic acid, catechol, and ferulic acid; and 0-20% lignin or lignin derived compounds.

### Example 20

In a prophetic example, ferulic acid of ≤60% purity is converted through biological means to produce a final product of vanillin. The vanillin product is contained in a unique product mixture where impurities from the initial ferulic acid are found in the vanillin product mixture. The conversion of ferulic acid to vanillin is enabled by biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and it is noted that those skilled in the art will be aware of other microorganisms and biological agents that can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system. The ≤60% pure ferulic acid used for this example contains 0-40% lignin or lignin derived compounds and 0-40% carbohydrates, hemicellulose or carbohydrate derived materials as contaminants. The product mixture contains 15-60% vanillin; 0-45% other bioproducts including but not limited to guaiacol, vanillic acid, vanillyl alcohol, 4-vinylguaiacol, protocatechuic acid, catechol, ferulic acid; and 0-40% lignin or lignin derived compounds; and 0-40% carbohydrates, hemicellulose or carbohydrate derived materials.

### Example 21

In a prophetic example, ferulic acid of ≤30% purity is converted through biological means to produce a final product of vanillin. The vanillin product is contained in a unique product mixture where impurities from the initial ferulic acid are found in the vanillin product mixture. The conversion of ferulic acid to vanillin is enabled by biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and it is noted that those skilled in the art will be aware of other microorganisms and biological agents that can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system. The ≤30% pure ferulic acid used for this example contains 0-70% lignin or lignin derived compounds and 0-50% carbohydrates, hemicellulose or carbohydrate derived materials as contaminants. The product mixture contains 7.5-30% vanillin; 0-22.5% other bioproducts including but not limited to guaiacol, vanillic acid, vanillyl alcohol, 4-vinylguaiacol, protocatechuic acid, catechol, and ferulic acid; and 0-70% lignin or lignin derived compounds; and 0-50% carbohydrates, hemicellulose or carbohydrate derived materials.

### Example 22

In a prophetic example, ferulic acid of ≤20% purity is converted through biological means to produce a final product of vanillin. The vanillin product is contained in a unique product mixture where impurities from the initial ferulic acid are found in the vanillin product mixture. The conversion of ferulic acid to vanillin is enabled by biological agents known to convert ferulic acid to vanillin including but not limited to: Pseudomonas sp., including Pseudomonas fluorescens, Pseudomonas fluorescens BF13-1p4, Pseudomonas putida, Pseudomonas putida KT2440, Amycolatopsis sp., including Amycolatopsis sp. ATCC 39116 (also known as Streptomyces setonii), Amycolatopsis sp. HR 167, Streptomyces sp., including, Streptomyces sp. Strain V-1, Pediococcus acidilactici, Enterobacter sp. Px6-4, Escherichia coli, Escherichia coli JM109/pBB1, Phanerochaete chrysosporium, Phanerochaete chrysosporium NCIM 1197, Pycnoporus cinnabarinus, and/or Aspergillus niger. This list of biological agents for the conversion of ferulic acid to vanillin is non-exhaustive and it is noted that those skilled in the art will be aware of other microorganisms and biological agents that can be used to complete the conversion of ferulic acid to vanillin. The microorganisms can be cultured using conventional culture media and methods in either a shake flask or a bioreactor using intermittent or continuous feed of substrate and other nutrients. The cultivation setup may include a solid phase consisting of agar, agarose, alginate, polyurethane foam or other similar suitable gel-like or solid material to support microbial growth. The cultivation setup may further include a resin for adsorption of substrate(s) and/or product(s), to facilitate feed and harvesting of product, and to increase flux through the system. The ≤20% pure ferulic acid used for this example contains 0-70% lignin or lignin derived compounds, 0-60% carbohydrates or carbohydrate derived materials, and 0-30% protein, amino acids, or derivatives of protein as contaminants. The product mixture contains 5-20% vanillin; 0-15% other metabolites including but not limited to guaiacol, vanillic acid, vanillyl alcohol, 4-vinylguaiacol, protocatechuic acid, and catechol; and 0-70% lignin or lignin derived compounds; and 0-60% carbohydrates or carbohydrate derived materials; and 0-30% protein, amino acids, or derivatives of protein.

Having described various processes and systems, certain aspect can include, but are not limited to:

In a first aspect, a process for a separation of organic molecules from biomass comprises contacting biomass with a base; extracting products from the biomass based on a reaction of the base with the biomass to form a slurry; filtering the slurry to recover a liquid phase and a solid phase, wherein the liquid phase comprises a product, and wherein the product comprises: a) a ferulate, a coumarate, ferulic acid, coumaric acid, or any combination thereof; and separating the product from the liquid phase.

A second aspect can include the method of the first aspect, where the biomass comprises water.

A third aspect can include the method of the first or second aspect, wherein the biomass has between 0.1 to 75 wt. % water.

A fourth aspect can include the method of any one of the first to third aspects, further comprising contacting the biomass with a solvent, wherein the solvent comprises water, an alcohol, acetone, an organic solvent, or any combination thereof.

A fifth aspect can include the method of any one of the first to fourth aspects, wherein the base has a concentration between about 0.01 M to about 6 M.

A sixth aspect can include the method of any one of the first to fifth aspects, wherein a ratio of the solvent to the biomass on a dry basis is between about 1:1 to about 30:1, or between about 1:1 to 25:1.

A seventh aspect can include the method of any one of the first to sixth aspects, further comprising: rinsing the solid product with a solvent comprising an alcohol, acetone, ether, organic solvent or any combination thereof to recover a second liquid phase, wherein the second liquid phase is added to the liquid phase.

An eighth aspect can include the method of any one of the first to third aspects, wherein separating the product from the liquid phase comprises: concentrating the liquid phase to a viscous liquid or semi-solid; and dissolving the viscous liquid or semi-solid in an organic solvent; forming an organic solvent phase, wherein the organic solvent phase comprises at least a portion of the product.

A ninth aspect can include the method of the eighth aspect, wherein a majority of one or more byproducts remain undissolved in the organic solvent.

A tenth aspect can include the method of the eighth or ninth aspects, wherein the organic solvent comprises at least one of hexane, heptane, ethyl acetate, acetone, an ether, an aliphatic alcohol, or any combination thereof.

An eleventh aspect can include the method of any one of the eighth to tenth aspects, further comprising: removing at least a portion of the organic solvent from the organic solvent phase to recover the product.

A twelfth aspect can include the method of any one of the first to eleventh aspects, wherein contacting the biomass with the base and the solvent occurs at a temperature between about 5 to about 200° C.

A thirteenth aspect can include the method of any one of the first to eleventh aspects, wherein contacting the biomass with the base and the solvent occurs at a temperature between about 10 to about 90° C., or at less than about 35° C., or at less or equal to 30° C.

A fourteenth aspect can include the method of the thirteenth aspect, wherein the liquid phase does not comprise phenolic lignin, or comprises less than 1% phenolic lignin molecules.

A fifteenth aspect can include the method of any one of the first to fourteenth aspects, further comprising: hydrolyzing the liquid phase prior to separating the product from the liquid phase.

A sixteenth aspect can include the method of the fifteenth aspect, wherein hydrolyzing the liquid phase comprises: using basic or acidic hydrolysis to hydrolyze arabinoxylans or hemicellulose within the liquid phase to arabinose, xylose, or any combination thereof.

A seventeenth aspect can include the method of the fifteenth or sixteenth aspect, further comprising: contacting the liquid phase with an organic solvent after the hydrolyzing; forming an organic solvent phase, wherein the organic solvent phase comprises at least a portion of the product.

An eighteenth aspect can include the method of any one of the first to seventeenth aspects, further comprising: adding an alcohol, an ether, acetone, hexane, heptane, an organic solvent or any combination thereof to the liquid phase; and precipitating arabinoxylans or hemicellulose from the liquid phase in response to adding the alcohol.

A nineteenth aspect can include the method of the eighteenth aspect, wherein the solvent is added to liquid phase in a concentration equal to or greater than 10 weight % or greater than 40 weight %.

A twentieth aspect can include the method of any one of the first to nineteenth aspects, further comprising: converting at least a portion of the product to vanillin.

In a twenty first aspect, a method of purifying a liquid product comprising ferulic acid or a ferulate, the method comprises providing a liquid product comprising an aqueous phase and a product, wherein the product comprises at least one of ferulic acid, a ferulate, or any combination thereof; and separating at least a portion of the product from the aqueous phase.

A twenty second aspect can include the method of the twenty first aspect, wherein separating at least the portion of the product comprises using concentration, selective dissolution, adsorption, hot water filtration, hot water filtration, organic solvent extraction, precipitation, sublimation, cation exchange, anion exchange, resin adsorption, ultrafiltration, microfiltration, nanofiltration or any combination thereof.

A twenty third aspect can include the method of the twenty first aspect, wherein separating at least the portion of the product comprises: using evaporation to concentrate the product in the aqueous phase to form a concentrate; and adjusting a pH of the concentrate to between about 1-5.

A twenty fourth aspect can include the method of the twenty third aspect, further comprising: contacting the concentrate with an organic solvent to form an organic phase and the aqueous phase; dissolving a majority of the product in the organic phase; and separating the organic phase from the aqueous phase; and removing at least a portion of the organic solvent from the organic phase to form a concentrated product mixture.

A twenty fifth aspect can include the method of the twenty fourth aspect, further comprising: passing the concentrated product mixture through a silica, alumina, cation exchange, anion exchange, or resin based column; flushing the column with a diluent; collecting the eluent from the column; and concentrating the eluent to form an enriched product mixture.

A twenty sixth aspect can include the method of the twenty fifth aspect, further comprising: combining the enriched product mixture with water to form a solution; heating the solution to above room temperature; forming a liquid portion and a solid precipitate in response to combining the enriched product mixture with the water and heating the solution; separating the solid precipitate from the liquid portion; cooling the liquid portion to below room temperature; forming a product precipitate in response to the cooling; and separating the product precipitate from the liquid portion, wherein the product precipitate comprises the product at a concentration of greater than 30%, 60%, or 80% purity.

A twenty seventh aspect can include the method of the twenty fourth aspect, further comprising: contacting the concentrated product mixture with an organic solvent to form an organic solvent phase as part of a two-phase mixture; extracting low polar impurities into the organic solvent phase to concentrate the product in the concentrated product mixture; and separating the organic solvent phase from the concentrated product mixture.

A twenty eighth aspect can include the method of the twenty seventh aspect, further comprising: combining the concentrated product mixture with water to form a solution; boiling the solution; forming a liquid portion and a solid precipitate in response to combining the enriched product mixture with the water and heating the solution; separating the solid precipitate from the liquid portion; cooling the liquid portion to below room temperature; forming a product precipitate in response to the cooling; and separating the product precipitate from the liquid portion, wherein the product precipitate comprises the product at a concentration of greater than 30%, 60%, or 80% purity.

A twenty ninth aspect can include the method of the twenty fourth aspect, further comprising: contacting the concentrated product mixture with an organic solvent; forming a three-phase mixture in response to the contacting, wherein the three phase mixture comprises: an organic solvent phase, a viscous phase; and a solid phase; filtering the solid phase from the three-phase mixture, wherein the solid phase comprises a portion of the product; separating the viscous phase from the organic solvent phase; contacting the viscous phase with a second organic solvent to form a second organic phase and a second solid phase; separating the second organic phase; concentrating the second organic phase by removing at least a portion of the second organic solvent to form a second product mixture; and combining the second product mixture and the solid phase to form a third product mixture.

A thirtieth aspect can include the method of the twenty ninth aspect, further comprising: combining the third product mixture with water to form a solution; boiling the solution; forming a liquid portion and a solid precipitate in response to combining the enriched product mixture with the water and heating the solution; separating the solid precipitate from the liquid portion; cooling the liquid portion to below room temperature; forming a product precipitate in response to the cooling; and separating the product precipitate from the liquid portion, wherein the product precipitate comprises the product at a concentration of greater than 30%, 60%, or 80% purity.

A thirty first aspect can include the method of the twenty fourth aspect, further comprising: contacting the concentrated product mixture with an organic solvent; forming a three-phase mixture in response to the contacting, wherein the three-phase mixture comprises: an organic solvent phase, a viscous phase; and a solid phase; filtering the solid phase from the three-phase mixture, wherein the solid phase comprises a portion of the product; separating the viscous phase from the organic solvent phase; contacting the viscous phase with a second organic solvent to form a second organic phase and a second solid phase; separating the second organic phase; and concentrating the second organic phase by removing at least a portion of the second organic solvent to form a second product mixture.

A thirty second aspect can include the method of the thirty first aspect, further comprising: passing the second product mixture through a gel column; flushing the gel column with a diluent; collecting the eluent from the gel column; and concentrating the eluent to form an enriched product mixture.

A thirty third aspect can include the method of the twenty first aspect, wherein separating at least the portion of the product comprises: using evaporation to concentrate the product in the aqueous phase to form a concentrate; passing the concentrate through a gel column; flushing the gel column with a diluent; collecting the eluent from the gel column; and concentrating the eluent to form an enriched product mixture.

A thirty fourth aspect can include the method of the thirty third aspect, further comprising: combining the enriched product mixture with water to form a solution; boiling the solution; forming a liquid portion and a solid precipitate in response to combining the enriched product mixture with the water and heating the solution; separating the solid precipitate from the liquid portion; cooling the liquid portion to below room temperature; forming a product precipitate in response to the cooling; and separating the product precipitate from the liquid portion, wherein the product precipitate comprises the product at a concentration of greater than 80% purity.

A thirty fifth aspect can include the method of the twenty first aspect, wherein separating at least the portion of the product comprises: using evaporation to concentrate the product in the aqueous phase to form a concentrate; cooling the concentrate to form a solid; forming a powder from the solid at a temperature below about 10 C or spray drying to form a solid; adding the powder to an aqueous fluid to form a mixture; boiling the mixture; forming a three-phase mixture in response to the boiling, wherein the three phase mixture comprises a liquid phase, a viscous phase, and a solid phase; separating the liquid phase from the three-phase mixture; cooling the liquid phase to form a precipitate; separating the precipitate from the liquid phase as a product.

A thirty sixth aspect can include the method of the thirty fifth aspect, further comprising: combining the liquid phase with an organic solvent; precipitating impurities from the liquid phase in response to combining the liquid phase with the organic solvent; and concentrating the liquid phase after precipitating the impurities.

A thirty seventh aspect can include the method of any one of the twenty first to thirty sixth aspects, wherein the liquid product is obtained from any of the methods of first to twentieth aspects.

A thirty eighth aspect can include the method of any one of the twenty first to thirty sixth aspects, further comprising: converting at least a portion of the product to vanillin.

A thirty ninth aspect can include the method of any one of the twenty first to thirty sixth aspects, wherein the liquid product is obtained from water used in the processing of organic materials that contain ferulic acid or ferulate.

A fortieth aspect can include the method of any one of the thirty first to thirty sixth aspects, where ferulic acid of ≤90% purity is converted to vanillin forming a product mixture comprising 22.5-90% vanillin; 0-67.5% bioproducts, wherein the bioproducts comprise at least one of guaiacol, vanillic acid, vanillyl alcohol, 4-vinylguaiacol, protocatechuic acid, catechol, and ferulic acid; and 0-10% lignin or lignin derived compounds.

A forty first aspect can include the method of any one of the thirty first to thirty sixth aspects, where ferulic acid of ≤80% purity is converted to vanillin forming a product mixture product containing 20-80% vanillin; 0-60% bioproducts comprising at least one of vanillic acid, vanillyl alcohol, 4-vinylguaiacol, protocatechuic acid, catechol, and ferulic acid; and 0-20% lignin or lignin derived compounds.

A forty second aspect can include the method of any one of the thirty first to thirty sixth aspects, where ferulic acid of ≤20% purity or up to 60% purity is converted to vanillin forming a product mixture product containing 5-60% vanillin; 0-45% bioproducts comprising at least one of guaiacol, vanillic acid, vanillyl alcohol, 4-vinylguaiacol, protocatechuic acid, catechol, and ferulic acid; 0-70% lignin or lignin derived compounds; 0-60% carbohydrates, hemicellulose or carbohydrate derived materials; and 0-30% protein, amino acids or protein derivatives.

In the preceding discussion and in the claims, the terms “including” and “comprising” are used in an open-ended fashion, and thus should be interpreted to mean “including, but not limited to . . . ”. At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rl, and an upper limit, Ru, is disclosed, any number falling within the range is specifically disclosed. In particular, the following numbers within the range are specifically disclosed: R=Rl+k*(Ru-Rl), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present invention.

1. A process for a separation of organic molecules from biomass comprising:
contacting biomass with a base;
contacting the biomass with a solvent, wherein the solvent comprises water, an alcohol, acetone, an organic solvent, or any combination thereof;
extracting products from the biomass based on a reaction of the base with the biomass to form a slurry;
filtering the slurry to recover a liquid phase and a solid phase, wherein the liquid phase comprises a product, and wherein the product comprises: a ferulate, a coumarate, ferulic acid, coumaric acid, or any combination thereof; and
separating the product from the liquid phase, wherein separating the product from the liquid phase comprises:
concentrating the liquid phase to a viscous liquid or semi-solid; and
dissolving the viscous liquid or semi-solid in an organic solvent;
forming an organic solvent phase, wherein the organic solvent phase comprises at least a portion of the product, and wherein a majority of one or more byproducts remain undissolved in the organic solvent.

2. (canceled)

3. The method of claim 1, further comprising: rinsing the solid product with a solvent comprising an alcohol, acetone, ether, organic solvent or any combination thereof to recover a second liquid phase, wherein the second liquid phase is added to the liquid phase.

4.-5. (canceled)

6. The method of claim 1, wherein the organic solvent comprises at least one of hexane, heptane, ethyl acetate, acetone, an ether, an aliphatic alcohol, or any combination thereof, and where the method further comprises:
removing at least a portion of the organic solvent from the organic solvent phase to recover the product.

7. (canceled)

8. The method of claim 1, further comprising:
hydrolyzing the liquid phase prior to separating the product from the liquid phase; and
using basic or acidic hydrolysis to hydrolyze arabinoxylans within the liquid phase to arabinose, xylose, or any combination thereof.

9.-10. (canceled)

11. The method of claim 1, further comprising:
adding an alcohol, an ether, acetone, hexane, heptane, an organic solvent or any combination thereof to the liquid phase; and
precipitating arabinoxylans or hemicellulose from the liquid phase in response to adding the solvent, wherein the solvent is added to the liquid phase in a concentration equal to or greater than 10 weight % or greater than 40 weight %.

12. (canceled)

13. The method of claim 1, further comprising:
converting at least a portion of the product to vanillin.

14. A method of purifying a liquid product comprising ferulic acid or a ferulate, the method comprising:
providing a liquid product comprising an aqueous phase and a product, wherein the product comprises at least one of ferulic acid, a ferulate, or any combination thereof; and
separating at least a portion of the product from the aqueous phase.

15. The method of claim 14, wherein separating at least the portion of the product comprises using concentration, selective dissolution, adsorption, hot water filtration, hot water filtration, organic solvent extraction, precipitation, sublimation, cation exchange, anion exchange, resin adsorption, ultrafiltration, microfiltration, nanofiltration or any combination thereof.

16. The method of claim 14, wherein separating at least the portion of the product comprises:
concentrating the product in the aqueous phase to form a concentrate; and
adjusting a pH of the concentrate to between about 1-5.

17. The method of claim 16, further comprising:
contacting the concentrate with an organic solvent to form an organic phase and the aqueous phase;
dissolving a majority of the product in the organic phase;
separating the organic phase from the aqueous phase; and
removing at least a portion of the organic solvent from the organic phase to form a concentrated product mixture.

18. (canceled)

19. The method of claim 17, further comprising:
combining the enriched product mixture with water to form a solution;
heating the solution to above room temperature;
forming a liquid portion and a solid precipitate in response to combining the enriched product mixture with the water and heating the solution;
separating the solid precipitate from the liquid portion;
cooling the liquid portion to below room temperature;
forming a product precipitate in response to the cooling; and
separating the product precipitate from the liquid portion, wherein the product precipitate comprises the product at a concentration of greater than 30%, 60%, or 80% purity.

20. The method of claim 17, further comprising:
contacting the concentrated product mixture with an organic solvent to form an organic solvent phase as part of a two-phase mixture;
extracting low polar impurities into the organic solvent phase to concentrate the product in the concentrated product mixture; and
separating the organic solvent phase from the concentrated product mixture.

21. The method of claim 20, further comprising:
combining the concentrated product mixture with water to form a solution;
boiling the solution;
forming a liquid portion and a solid precipitate in response to combining the enriched product mixture with the water and heating the solution;
separating the solid precipitate from the liquid portion;
cooling the liquid portion to below room temperature;
forming a product precipitate in response to the cooling; and
separating the product precipitate from the liquid portion, wherein the product precipitate comprises the product at a concentration of greater than 30%, 60%, or 80% purity.

22. (canceled)

23. The method of any of claim 14, further comprising:
converting at least a portion of the product to vanillin.

24. The method of any one of claim 14, wherein the liquid product is obtained from water used in the processing of organic materials that contain ferulic acid or ferulate.

25. The method of any of claim 14, where ferulic acid of ≤90% purity is converted to vanillin forming a product mixture comprising 22.5-90% vanillin; 0-67.5% bioproducts, wherein the bioproducts comprise at least one of guaiacol, vanillic acid, vanillyl alcohol, 4-vinylguaiacol, protocatechuic acid, catechol, and ferulic acid; and 0-10% lignin or lignin derived compounds.

26. The method of any of claim 14, where ferulic acid of ≤80% purity is converted to vanillin forming a product mixture product containing 20-80% vanillin; 0-60% bioproducts comprising at least one of vanillic acid, vanillyl alcohol, 4-vinylguaiacol, protocatechuic acid, catechol, and ferulic acid; and 0-20% lignin or lignin derived compounds.

27. The method of any of claim 14, where ferulic acid of ≤20% purity or up to 60% purity is converted to vanillin forming a product mixture product containing 5-60% vanillin; 0-45% bioproducts comprising at least one of guaiacol, vanillic acid, vanillyl alcohol, 4-vinylguaiacol, protocatechuic acid, catechol, and ferulic acid; 0-70% lignin or lignin derived compounds; 0-60% carbohydrates, hemicellulose or carbohydrate derived materials; and 0-30% protein, amino acids or protein derivatives.

28. The method of claim 14, wherein the product has a concentration of ferulic acid between 15 wt. % to 100%, and wherein the method further comprises:
converting at least a portion of the ferulic acid in the product to vanillin using the biological agent of the genus Amycolatopsis or Streptomyces to produce a vanillin mixture.

29. The method of claim 28 where the vanillin mixture comprises one or more of vanillin, guaiacol, vanillic acid, vanillyl alcohol, 4-vinylguaiacol, protocatechuic acid, catechol, ferulic acid, lignin or lignin derived compounds, carbohydrates, hemicellulose, carbohydrate derived materials, a protein, amino acids, protein derivatives, or an oil.
